(19) United States
(12) Patent Application Publication
(10) Pub. No.: US 2024/0077482 A1
(43) Pub. Date: Mar. 7, 2024

(54) METHODS AND APPARATUSES FOR DETECTING RESPIRATORY INFECTIONS

(71) Applicant: ControlPoint Inc., Campbell, CA (US)

(72) Inventors: Paula Walker, Campbell, CA (US); Johnny Callahan, Monrovia, MD (US)

(21) Appl. No.: 18/296,982

(22) Filed: Apr. 6, 2023

Related U.S. Application Data
(63) Continuation of application No. PCT/US2021/054039, filed on Oct. 7, 2021.
(60) Provisional application No. 63/089,523, filed on Oct. 8, 2020.

(51) Int. Cl. G01N 33/569

(52) U.S. Cl. CPC G01N 33/56983; G01N 33/56911; G01N 2800/26

(57) ABSTRACT

Methods and devices for detecting and diagnosing the etiological agents associated with Canine Infectious Respiratory Disease Complex in animals, such as kennel cough, are disclosed herein.

### CROSS-REFERENCE

This application is a continuation application of International Patent Application No. PCT/US2021/054039, filed Oct. 7, 2021, which claims priority to U.S. Provisional Application No. 63/089,523, filed Oct. 8, 2020, each of which is hereby incorporated by reference in its entirety.

### BACKGROUND

Kennel cough, also known as canine infectious respiratory disease complex (CIRDC) or canine infectious tracheobronchitis (ITB), is an upper respiratory infection affecting dogs. CIRDC is considered one of the most prevalent infectious respiratory diseases of dogs worldwide, and outbreaks can reach epidemic proportions when dogs are housed in high-density population environments, such as kennels. The range of etiologic agents includes viruses and bacteria, such as Canine adenovirus type-2 (CAV-2), Canine parainfluenza virus (CPIV), Canine coronavirus (CCoV), canine herpesvirus-1 (CHV), and most commonly, the bacterium Bordetella bronchiseptica (Bb). Infection with kennel cough predisposes dogs to secondary infections, such as viral infections that colonize the epithelium of the upper and lower respiratory tract frequently leading to more severe, sometimes fatal respiratory distress.

The range of etiologic agents includes viruses and bacteria that are detected and differentiated by specific laboratory tests. Due to the lack of commercially available diagnostic tests, many of the disease agents associated with CIRDC remain unrecognized and underreported.

Despite the prevalence of vaccinations, CIRDC remains a problem due, for example, to the rapid transmission of the underlying infection between animals and the resulting increase in susceptibility to secondary, more severe respiratory infections. Moreover, the lapse in immunity to the infection can occur even among previously vaccinated animals amplifying the need to evaluate the immune status of animals to the infectious agents causing CIRDC. Therefore, there remains a considerable need for methods and devices for detecting CIRDC, especially at the early stages of disease or infection, in order to provide guidance on treatment or intervention to reduce the progression or transmission of CIRDC.

### SUMMARY

In some aspects, the present disclosure provides a method for processing or analyzing a sample from a subject in need thereof, the method comprising processing the sample using a lateral flow device configured to specifically bind to one or more target analytes associated with a respiratory condition associated with one or more pathogens that can cause Canine Infectious Respiratory Disease Complex (CIRDC) in one or more discrete sections of the lateral flow device, wherein the lateral flow device is configured to produce a detectable signal in the one or more discrete sections upon binding to the one or more target analytes; and analyzing the detectable signal to determine if the subject has the respiratory condition or has an immunity to the respiratory condition. The method can comprise obtaining the sample from the subject via swab. The sample can be selected from the group consisting of saliva, ocular discharge, nasal discharge, oropharyngeal fluid, oral rinse expectorant, oral fluid, gingival crevicular fluid, urine, sweat, tears, blood, serum, stool, gastric fluid, synovial fluid, and phlegm. The target analytes can comprise one or more proteins. The one or more proteins can be an antigen produced by a CIRDC pathogen, a virulence factor produced by the CIRDC pathogen, a fragment of a virulence factor produced by the CIRDC pathogen, the CIRDC pathogen or an immunogenic fragment thereof. The CIRDC pathogen can be selected from the group consisting of Bordetella brochiseptica; Canine parainfluenza virus; Canine coronavirus; Canine adenovirus type-2; Canine herpesvirus; Canine distemper virus; Canine Influenzavirus; Canine Pneumovirus; Mycoplasma cynos; Streptococcus equi; Canine bocavirus; and Canine hepacivirus. The antigen can be a virulence factor of one or more of: Bordetella brochiseptica, canine parainfluenza virus, canine respiratory coronavirus, canine adenovirus type-2, canine herpesvirus, canine distemper virus, canine influenza virus, canine pneumovirus, Mycoplasma cynos, Streptococcus equi, and canine bocavirus. The virulence factor can be selected from the group comprising: a filamentous hemagluttinin protein (FHA), a pertactin protein, a branched chain fatty acid protein, a hemagglutinin protein, a fusion protein, a matrix protein, a spike glycoprotein trimer protein, a membrane protein, a nucleoprotein, an envelope small membrane pentramer protein, a capsid protein, a fiber protein, a penton protein, a hexon protein, an envelope protein, a capsid protein, a hemagglutinin or a fusion protein thereof, a matrix protein, an H3 protein, a N2 protein, a N8 protein, a glycoprotein, a SH protein, a microtubule associated protein (MAP), a carbamoyl phosphate synthetase (CPS) protein, a cerulopasmin (Cp) protein, or an immunogenic fragment thereof. The target analytes can comprise one or more target antibodies. The one or more target antibodies can comprise antibodies with affinity to one or more immunogenic components of a CIRDC pathogen. The target antibodies can be selected from the group consisting of an antibody with affinity to a virulence factor of one or more of: Bordetella brochiseptica, canine parainfluenza virus, canine respiratory coronavirus, canine adenovirus type-2, canine herpesvirus, canine distemper virus, canine influenza virus, canine pneumo virus, Mycoplasma cynos, Streptococcus equi, and canine bocavirusproteins. The virulence factor can be selected from the group comprising: a filamentous hemagluttinin protein (FHA), a pertactin protein, a branched chain fatty acid protein, a hemagglutinin protein, a fusion protein, a matrix protein, a spike glycoprotein trimer protein, a membrane protein, a nucleoprotein, an envelope small membrane pentramer protein, a capsid protein, a fiber protein, a penton protein, a hexon protein, an envelope protein, a capsid protein, a hemagglutinin or a fusion protein thereof, a matrix protein, an H3 protein, a N2 protein, a N8 protein, a glycoprotein, a SH protein, a microtubule associated protein (MAP), a carbamoyl phosphate synthetase (CPS) protein, a cerulopasmin (Cp) protein, or an immunogenic fragment thereof. The vaccination status of the subject for the respiratory condition can be unknown. The subject can be a non-human animal. The subject can belong to a genus selected from the group consisting of Canis familiaris, Sus scrofa domesticus, Fells catus, Oryctolagus cuniculus and Equus caballus. The subject can be a dog. The respiratory condition can be an infectious tracheobronchitis. The infectious tracheobronchitis can be a Bordetella brochiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus or Canine hepacivirus infection. The infectious tracheobronchitis can be a viral infection. The infectious tracheobronchitis can be a bacterial infection. The method can comprise evaluating an onset of protective immunity to the respiratory condition in the subject that has received a vaccine to the respiratory condition. Analyzing can comprise comparing the detectable signal to a control to determine a presence of the one or more target analytes in the sample. Comparing can comprise analyzing a digital image of the detectable signal produced by the lateral flow device, wherein the digital image comprises a code to identify the control. Analyzing can be performed by a trained algorithm, wherein the digital image is sent to the trained algorithm via a computer. The method can comprise obtaining a second sample from the subject at a second time point and analyzing the second sample using the lateral flow device to evaluate a duration of immunity to the respiratory condition. The lateral flow device can comprise one or more capture agents. The one or more capture agents can comprise one or more antigens associated with the respiratory condition. The one or more capture agents can comprise one or more antibodies configured to bind an antigen associated with the respiratory condition.

Disclosed herein is a system for processing or analyzing a sample from a subject in need thereof comprising a lateral flow device comprising: (i) two or more assay strips, each comprising two or more discrete reaction zones on a solid support, wherein a first discrete reaction zone comprises a control analyte capture agent and a second discrete reaction zone comprises a target analyte capture reagent; wherein the target analyte capture reagent is a Canine Infectious Respiratory Disease Complex (CIRDC) antigen specific antibody, a CIRDC antigen, or a fragment thereof; and (ii) a sample input port wherein the two or more assay strips are positioned around the sample input port; and a computer, wherein the lateral flow device is configured to produce a detectable signal upon binding of the control analyte capture agent to a control analyte or upon binding of the target analyte capture agent to a target analyte, wherein the detectable signal is sent to the computer and analyzed by a computer program to determine a presence or absence of a CIRDC antigen, antibody or fragment thereof. The computer can output a likelihood that the subject has a respiratory condition, will develop the respiratory condition, or has an immunity to the respiratory condition. The system can further comprise a first reporter molecule configured to bind to a control analyte bound by the control analyte capture agent and a second reporter molecule configured to bind to a target analyte bound by the target analyte capture agent. The first reporter molecule and the second reporter molecule can be capable of producing the detectable signal in a discrete reaction zone of the two or more discrete reaction zones upon binding to the target analyte or the control analyte. The first reporter molecule or the second reporter molecule can comprise colloidal gold, protein A, an enzyme, or an indicator dye. The detectable signal can be a fluorescent signal or a colorimetric change. The biological sample can be selected from the group consisting of saliva, nasal discharge, oral discharge, oropharyngeal fluid, oral rinse expectorant, oral fluid, gingival crevicular fluid, urine, sweat, tears, blood, serum, stool, gastric fluid, synovial fluid, and phlegm. The control analyte capture agent can be an antibody and the target analyte capture agent is an antibody. The control analyte capture agent can be IgA. The control analyte can be an antigen and the target analyte can be an antigen. The target analyte can be a virulence factor of Bordetella bronchiseptica, Canine parainfluenza virus, Canine coronavirus, Canine adenovirus type-2, Canine herpesvirus, Canine distemper, Canine Influenza, Canine Pneumovirus, Mycoplasma cynos, Streptococcus equi, Canine bocavirus or Canine hepacivirus. The virulence factor can be selected from the group comprising: a filamentous hemagluttinin protein (FHA), a pertactin protein, a branched chain fatty acid protein, a hemagglutinin protein, a fusion protein, a matrix protein, a spike glycoprotein trimer protein, a membrane protein, a nucleoprotein, an envelope small membrane pentramer protein, a capsid protein, a fiber protein, a penton protein, a hexon protein, an envelope protein, a capsid protein, a hemagglutinin or a fusion protein thereof, a matrix protein, an H3 protein, a N2 protein, a N8 protein, a glycoprotein, a SH protein, a microtubule associated protein (MAP), a carbamoyl phosphate synthetase (CPS) protein, and a cerulopasmin (Cp) protein. The target analyte can be a fragment of a virulence factor produced by Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus, Canine hepacivirus, or Canis familiaris. The virulence factor can be a structural protein, cell membrane protein or toxin produced by Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus or Canine hepacivirus in a Canis familiaris animal. The control analyte capture agent can be an antigen and the target analyte capture agent is an antigen. The control analyte can be an antibody and the target analyte can be an antibody. The control analyte capture agent can be canine IgA. The control analyte can be an anti-IgA antibody. The target analyte can be an antibody selected from the group consisting of an antibody with affinity to a CIRDC pathogen or an immunogenic fragment thereof. The CIRDC pathogen can be Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus or Canine hepacivirus. The target analyte can be an antibody selected from the group consisting of antibodies with affinity to a virulence factor of the group consisting of: Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus and Canine hepacivirus. The lateral flow device can comprise an immunochromatographic assay strip. The immunochromatographic assay strip can comprise a nitrocellulose membrane. The immunochromatographic assay strip can be configured to form a labeled capture agent-control analyte conjugate in the first discrete reaction zone and a labeled capture agent-target analyte conjugate in the second discrete reaction zone.

Disclosed herein is a device for detecting one or more target analytes associated with one or more respiratory conditions, wherein the device comprises one or more target analyte capture reagents immobilized on a solid support; wherein the one or more target analyte capture reagents are a Canine Infectious Respiratory Disease Complex (CIRDC) antigen specific antibody, a CIRDC antigen, or a fragment thereof; wherein each of the one or more target analyte capture reagents is in a different discrete section of a plurality of discrete sections of the device; wherein the plurality of discrete sections are fluidly connected to a central sample input port; and wherein the device is configured to produce a detectable signal upon binding of the one or more target analytes by the one or more target analyte capture agents. The solid support can comprise an immunochromatographic assay strip. The immunochromatographic assay strip can comprise nitrocellulose. The plurality of target and control analyte discrete sections can be positioned in one or more strips circular manner around the central sample input port. The first control analyte capture section and the first target analyte capture section can be positioned on a first strip and the second control analyte capture section and the second target analyte capture section can be positioned on a second strip, wherein the first strip and the second strip are positioned around the central sample input port. The plurality of discrete sections can be present in a single strip wherein the sections are positioned distally to the central sample input port. The plurality of discrete sections can be positioned such that the target analyte sections are arranged as a two-dimensional array on the assay strip distal to the sample input port. The plurality of discrete sections can be positioned such that each discrete target analyte section is positioned adjacent to a discrete control analyte section. The plurality of discrete sections can be positioned such that each discrete target analyte section is positioned adjacent to another target analyte section. The target analyte section can comprise a single capture reagent. The target analyte section can comprise a combination of capture reagents. The device can comprise at least 12 discrete target analyte sections wherein a presence or absence of each target analyte indicates a presence or absence of a different CIRDC pathogen. The CIRDC pathogen can be selected from the group consisting of Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus or Canine hepacivirus. The immunochromatographic assay strip can be removable.

The plurality of target and control analyte reaction sites on the solid phase could alternatively be positioned as discrete “dots” on a single nitrocellulose strip within a single cartridge. Each dot location represents a specific antigen capture reaction comprised of a monoclonal capture antibody, an antigen analyte, and a conjugated detector antibody with a colloidal gold indicator unique to one of the target organisms and to one of their protein antigens. For example, the Bordetella bronchiseptica organism could have three specific target proteins including the filamentous hemagglutinin (FHA); Pertactin (PRN); and Bordetella Colonization Factor A (Bcfa) antigen targets. In this inception, the goal is to align more than one detection site in a single analytical device. In this case, a specific monoclonal antibody with specificity to target protein is applied to the nitrocellulose strip as discrete dot to serve as a capture antibody. If the target analyte is present in the animal sample, that antigen is captured to form an immune complex. Next, a detector antibody conjugate using colloidal gold is used to create a reaction that can be read with a device. To screen for 12 pathogens containing three distinct protein analytes each, 36 discrete dots could be arranged in an array-like format where any suitable configuration such as a six by six, or four by nine array could be used (FIG. 6.)

Disclosed herein is a kit comprising the device and instructions. The kit can comprise an apparatus for obtaining a sample from a subject and a transport fluid for stabilizing a sample prior to loading onto the sample input port of the device. The kit can comprise a buffer to produce the detectable signal. The buffer can comprise a signal generating reagent. The transport fluid can comprise a protease inhibitor and ethylenediaminetetraacetic acid (EDTA).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of a biphasic response to a CIRDC infection.

FIG. 2 shows a schematic of a portable lateral-flow device containing an immunochromatographic assay strip that can be used to test for the presence of one or more CIRDC pathogens or one or more immunogenic components thereof in a biological fluid obtained from an animal, and thereby for the presence of the infection.

FIG. 3 shows a schematic of a portable lateral-flow device containing an immunochromatographic assay strip that can be used to test for the presence of one or more antibodies that are generated in response to CIRDC pathogens upon infecting an animal. The device uses biological fluid from the animal and the test tests for the immunization status of the animal to one or more CIRDC pathogens.

FIG. 4 shows a schematic of the steps involved in multiplexed detection of etiological agents that can cause CIRDC infection using a device as disclosed herein.

FIG. 5 shows a schematic of an example of a device as disclosed herein.

FIG. 6A shows a schematic of an example of a device as disclosed herein. FIG. 6B shows the inner components of device disclosed herein.

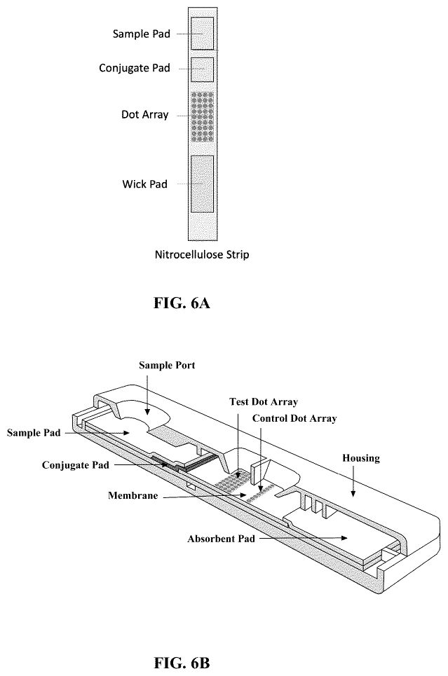

FIG. 7 shows an example of a backend structure for an application database capable of storing and analyzing diagnostic data.

### DETAILED DESCRIPTION

Described herein are devices, systems, kits and methods for detecting the presence of a target pathogen, such as a pathogen that can cause CIRDC in a biological sample. In the following disclosure, a CIRDC pathogen can be, for example, Bordetella brochiseptica, Canine parainfluenza virus, Canine coronavirus, Canine adenovirus type-2, Canine herpesvirus, Canine distemper virus, Canine Influenzavirus, Canine Pneumovirus, Mycoplasma cynos, Streptococcus equi, Canine bocavirus, Canine hepacivirus, etc. The devices, systems, kits and methods can be used in a rapid test (e.g., lab test or point-of-care test) for the detection of the target pathogen of interest, such as a CIRDC pathogen (e.g., Bordetella bronchoseptica), in a sample derived from or obtained from an animal, such as a dog. The devices, systems, kits and methods disclosed herein may perform multiplexed detection of a one or more CIRDC pathogens (e.g., Bordetella bronchiseptica or Canine adenovirus type-2) infection. In some aspects, the present disclosure provides devices, systems, kits and methods for the early detection of a CIRDC infection in an animal such as a dog. Early detection of CIRDC or kennel cough may protect the animal from increased susceptibility to secondary respiratory infections caused by other disease agents. In some cases, the secondary infection is caused by Canine adenovirus type-2 (CAV-2), Canine parainfluenza virus (CPIV), Canine coronavirus (CCoV), Canine herpesvirus-1 (CHV) or Pasteurella multocida.

In some aspects, the present disclosure provides devices, systems, kits and methods for evaluating the immunization status of an animal to agents that can cause CIRDC (e.g., Canine Pneumovirus). The devices, systems, kits and methods may be used to evaluate the onset of protective immunity to one or more CIRDC pathogens (e.g., Bordetella bronchiseptica) in an animal following administration of a vaccine to one or more CIRDC pathogens (e.g., Bordetella bronchiseptica) or to evaluate the duration of immunity to one or more CIRDC pathogens (e.g., Bordetella bronchiseptica) in an animal following the administration of a vaccine to one or more CIRDC pathogens. The devices, systems, kits and methods disclosed herein aid in ensuring continued protection of the animal (e.g., dog), from one or more CIRDC pathogens (e.g., Bordetella bronchiseptica) infection. The devices, systems, kits and methods may reduce the rapid transmission of one or more CIRDC pathogens infection in a population of animals (e.g., dogs) by preventing or limiting a lapse in immunity to one or more agents that can cause CIRDC infection.

The present disclosure provides an immunoassay that will serve as a marker of vaccine status to a CIRDC pathogen (e.g., Streptococcus equi) to assess the level of protective antibodies. Applications of this test method may also provide useful data to determine the optimum number of doses and schedule for vaccinations to a CIRDC pathogen (e.g., Bordetella bronchoseptica). The methods disclosed herein may follow the progression of antibody response at each stage of a vaccination series to a CIRDC pathogen (e.g., Bordetella bronchoseptica). By monitoring an antibody titer, the methods disclosed herein may determine, for example, if an injection series is needed for vaccine effectiveness; if annual boosters are required to maintain protective immunization, or alternatively, how long the immunization lasts; and/or the point in the injection series at which maximum antibody titer is achieved.

In another aspect, the devices, systems, kits and methods herein can be applicable in distinguishing between viral and bacterial causative agents of respiratory distress in a symptomatic or acutely ill animal, as well as in evaluating the recovery of the animal from the disease. In one aspect, the methods described herein are directed towards obtaining a biological fluid (i.e., nasal swab) from the animal (i.e., dog) and rapidly detecting one or more CIRDC pathogens (e.g., Bordetella bronchiseptica). In another aspect, the methods disclosed herein provide the rapid detection of one or more antibodies to a CIRDC pathogen (e.g. Streptococcus equi) in a biological fluid (i.e., saliva), obtained from an animal (i.e., dog). In some cases, the assay may be conducted on a solid support (i.e., immunochromatographic strip) comprising surface-immobilized capture reagents (i.e., a labeled antibody with affinity to the Streptococcus equi antigen of interest). The presence of the antigen or antibody in the sample is detected by the generation of a signal that can then be measured and analyzed rapidly at the same location using a hand-held scanner.

The present disclosure is particularly useful as a rapid, sensitive, specific, non-invasive diagnostic screening tool to assess CIRDC pathogen exposure or immunization status of animals, particularly dogs. The hand-held, in vitro test method of the present disclosure may be used for surveillance of the health status of animals in training and housing areas where outbreak of CIRDC may be more likely due to the population and proximity of animals.

The devices, systems, kits and methods disclosed herein therefore address the various problems associated with diagnosing and evaluating transmission of CIRDC pathogen (e.g., Bordetella bronchiseptica) infection among animals such as dogs, averting the distress and economic losses incurred thereby. The resulting data can be used to generate laboratory reports or used by veterinary epidemiologists to fill knowledge gaps in the ecology, prevalence, incidence, and geographic distributions of agents contributing to CIRDC, that up until now, have been underrecognized and poorly understood.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of can include determining the amount of something present in addition to determining whether it is present or absent depending on the context.

The terms “capture reagent”, “capture agent” and “capture molecule” are used interchangeably herein.

The terms “target reaction zone” and “target analyte capture zone” are used interchangeably herein.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of clinical signs or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Antigen-Antibody Complexes

The present disclosure includes methods for using one or more of the devices, kits and/or compositions described here to detect the presence or absence of one or more CIRDC pathogens or one or more immunogenic antigens thereof (e.g., Bordetella bronchiseptica antigen) or antibodies to one or more CIRDC pathogens in a sample (e.g. bodily fluid) from a subject, such as an animal. In some embodiments, the subject is a dog.

In some embodiments, the subject has one or more symptoms of a respiratory condition, for example, serious or mucopurulent nasal, oral or ocular secretion, sneezing, coughing, high body temperature, retching, gagging, tachypnea, respiratory distress, systemic illness, lethargy, and decreased appetite. In some embodiments, the subject was not previously diagnosed with kennel cough. In some embodiments, the subject was previously diagnosed with kennel cough.

The methods herein may be carried out to detect the presence or absence of CIRDC pathogens or one or more immunogenic antigens thereof or antibodies to one or more CIRDC pathogens in a sample obtained from a domestic animal. In some embodiments, the methods detect the presence or absence of a CIRDC pathogen or one or more immunogenic antigens thereof, or antibody to a CIRDC pathogen in a sample obtained from a Canis familiaris, Sus scrofa domesticus, Felts catus, Oryctolagus cuniculus and Equus caballus animal. Samples can also be obtained from Macaca mulatta (rhesus monkey), M. fascicularis, M. nemestrina, Chlorocebus aethiops, Papio spp., Saimiri sciureus or Aotus trivirgatus.

The methods herein may be modified to detect the presence or absence of B. bronchiseptica, B. pertussis, b. parapertussis, B. hinzii, B. ansorpii, B. avium, B. bronchialis, B. flabilis, B. holmesii, B. muralis, B. petrii, B. pseudohinzii, B. sputigena, B. trematum, B. tumbae or B. tumicola. In some embodiments, the method comprises detect the presence or absence of B. bronchiseptica. The methods herein may also be used to detect the presence or absence of Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus, or Canine hepacivirus.

Provides herein are methods for detection of a respiratory infection in an animal, wherein the method detects one or more antigens present in a sample selected from one or more bodily fluids of the subject. In some embodiments, the method detects one or more CIRDC pathogens or one or more immunogenic antigens thereof (e.g., Bordetella bronchiseptica) present in a sample selected from one or more bodily fluids of the animal. The bodily fluid may be saliva, blood (e.g., whole blood, serum, buffy coat (i.e., mononuclear cells)), urine, feces, tissue, wound exudate, abscess material or mucus. In some embodiments, the bodily fluid is saliva. The bodily fluid may be from a nasal, oral and conjunctiva! sample. The sample may be collected with a swab and it may be added to a collection tube that will have approximately 0.5 mL, 1 mL, 1.5 mL, 2 mL, 2.5 mL, or 3 mL of media. Performance data for each sample type may be kept separate or be pooled. In some embodiments, the bodily fluid samples are added to the same collection tube. In some embodiments, the sample is a fresh sample. In some embodiments, the sample was previously frozen or previously stored with stabilizers. Stabilizers can include (1) sugars such as sucrose, trehalose, sorbitol and mannitol, or combination of sugars, or sugar alcohols such as glycerine; (2) methods that include sugars, such as lyophilization; vacuum centrifugation; crystallization or vitrification processes with the addition of any of the sugars mentioned, or with any combination of sugars; (3) blocking solutions like dried milk or milk derivatives, may remove surfactants or detergents, or lower their concentrations on membranes which impacts the wetting characteristics of the nitrocellulose strip; (4) Proteins like albumin or BSA and detergents like SDS or Tween 20 may promote resolubilization of the conjugate and may reduce non-specific binding between the reagents and the analyte; (5) High pH samples like urine, likely need pretreatment in high concentration buffer salts which can shift the sensitivity and specificity of the capture and detector antibodies (e.g. I. OM borate buffer, pH 9.5); (6) Use of methanol, ethanol, or isopropanol can improve the consistency of reagent application, decreases protein solubility, and enhances drying and fixation of proteins. When the sample under test for CIRDC contains soluble and insoluble matter, the soluble portion of the bodily fluid may be collected by any protocol known in art. The soluble portions of the sample generally may be collected by using filtration, extraction, centrifugation, or simple mixing followed by gravimetric settling. For example, the sample may be a bodily fluid that is naturally excreted or otherwise released by the mammal or that is artificially obtained from the mammal. Such artificial extraction may be carried out by milking the mammal or by injecting a syringe into the mammal and drawing the fluid into the syringe. Once obtained, the fluid optionally may be fractionated (for example, serum may be fractionated from whole blood as then used as the sample). As another example, the sample may be obtained by swabbing the mammal, such as the oral or nasal cavity of the mammal, for example. As yet another example, tissue sections may be obtained by biopsy.

In some embodiments, saliva and/or oral mucosal transudate and gingival crevicular fluid are mixed with buffer. Addition may be performed with a bulb pipette to ensure optimum membrane flow rate as the sample migrates the entire length of the membrane. Stimulated saliva and protease inhibitor/EDTA dilution buffer may be mixed to facilitate sample flow. In one embodiment, 4 drops of saliva and/or oral mucosal transudate and gingival crevicular fluid mixed with 2 drops of buffer, as added by bulb pipette may be added to ensure optimum membrane flow rate as the sample migrates the entire length of the membrane. Four drops (160-200 μL) of stimulated saliva mixed with 2 drops (80-100 μL) of protease inhibitor/EDT A dilution buffer may also facilitate sample flow.

Provided herein are methods to detect the presence or absence of a CIRDC pathogen or one or more immunogenic antigens thereof (e.g., Bordetella bronchiseptica antigen) in a biological fluid obtained from an animal as a marker of CIRDC infection. In one embodiment, the steps in the method include: collecting a sample of one or more bodily fluids from the animal; applying the sample to a solid support comprising at least one antibody with affinity to a CIRDC antigen or portion thereof in the sample, allowing the formation of at least one labeled antigen-antibody conjugate, wherein the conjugate is capable of generating a signal; allowing the signal to generate to detectable levels; and detecting the signal, wherein the presence of the antigen in the sample is determined by the presence or absence of the signal.

In some cases, the pathogen detected may be Bordetella bronchiseptica. In some cases, the pathogen detected may be Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus, or Canine hepacivirus.

In some cases, the antigen used as a marker for CIRDC infection in the assay may be a virulence factor produced by the pathogen in the animal (e.g., dog) or a fragment thereof. In some cases, the virulence factor is a structural protein, cell membrane protein or toxin produced by the pathogen. In some cases, the antigen that is used for detection of the CIRDC infection may be an immunogenic protein produced by the pathogen in the animal but not a virulence factor.

In some cases, the antigen that is detected may be a p68 protein, filamentous hemagglutinin (FHA) protein, pertactin (PER) protein, fimbriae (FIM) protein, Bordetella colonization factor A (Bcfa), tracheal colonization factor, serum resistance factor (BrkA), Bvg8 protein, O-antigen protein, adenylate cyclase-hemolysin, dermonecrotic toxin or tracheal cytotoxin of Bordetella bronchiseptica.

In some cases, the immunogenic fragment or antigen that is detected may be a Matrix (M), Hemagglutinin-neuraminidase protein (HN), Fusion protein (F), Nucleoprotein (NP), or the Matrix protein (M) of Canine parainfluenza virus. In some cases, the immunogenic fragment or antigen that is detected may be Spike glycoprotein trimer protein (S), Membrane protein (M), Nucleoprotein (N), or Envelope (E), small membrane pentamer protein of the Canine respiratory coronavirus. In some cases, the immunogenic fragment or antigen that is detected may be Capsid protein (C), Fiber protein, Penton protein or Peripentonal Hexon protein (PH), or Hexon (H) of the canine adenovirus type-2. In some cases, the immunogenic fragment or antigen that is detected may be the Envelope protein (E), Major capsid proteins (C), the Outer/Inner tegument proteins (T) of the Canine herpesvirus. In some cases, the immunogenic fragment or antigen that is detected may be the Hemagglutinin (H), Nucleocapsid (N), or the Fusion protein (F) of the Canine distemper virus. In some cases, the immunogenic fragment or antigen that is detected may be the Matrix Protein, Nucleoprotein (NP), H3 protein, N2 protein or N8 protein of the Canine influenzavirus. In some cases, the immunogenic fragment or antigen that is detected may be the Glycoprotein (G), Fusion protein (F), Matrix protein (M) or SH protein of the Canine Pneumovirus. In some cases, the immunogenic fragment or antigen that is detected may be MAP protein or CPS proteins of Streptococcus equi. In some cases, the immunogenic fragment or antigen that is detected may be Cp protein of Canine bocavirus. In some cases, the immunogenic fragment or antigen that is detected may be an E1 or E2 protein of Canine hepacivirus.

In some cases, an antibody that is used for detection of the CIRDC infection in the animal (e.g., dog) may be an antibody with specific affinity to an antigen produced by the pathogen upon infecting the animal (e.g., dog). In some cases, the antibody displays an affinity to a protein that is a virulence factor produced by the pathogen. In one example, the antibody displays an affinity to an immunogenic protein produced by the pathogen upon infecting the animal (e.g., dog).

In some cases, the antibody that has specific affinity to Bordetella bronchiseptica Pertactin may be used for detection of the infection. In one example, the antibody that is used for detection of the Bordetella bronchiseptica infection in the assay may be an antibody with specific affinity to Bordetella bronchiseptica Filamentous Hemagglutinin. In some cases, an antibody that displays specific affinity to Bordetella bronchiseptica O-antigen protein may be used in the assay.

In some cases, the antibody that is used for detection of the CIRDC infection may be an antibody with affinity to an immunogenic fragment of the Hemagglutinin protein (HN), Fusion protein (F) or the Matrix protein (M) of the Canine parainfluenza virus or immunogenic fragment thereof. In some cases, the antibody that is used for detection of the CIRDC infection may be an antibody with affinity to an immunogenic fragment of the Spike glycoprotein trimer protein, Membrane protein, Nucleoprotein or Envelope small membrane pentamer protein of the Canine respiratory coronavirus. In some cases, the antibody that is used for detection of the CIRDC infection may be an antibody with affinity to an immunogenic fragment of the Capsid protein, Fiber protein, Penton protein or Hexon protein of the canine adenovirus type-2. In some cases, the antibody that is used for detection of the CIRDC infection may be an antibody with affinity to an immunogenic fragment of the Envelope protein or Major capsid proteins of the Canine herpesvirus. In some cases, the antibody that is used for detection of the CIRDC infection may be an antibody with affinity to an immunogenic fragment of the Hemagglutinin or the Fusion protein of the Canine distemper virus. In some cases, the antibody that is used for detection of the CIRDC infection may be an antibody with affinity to an immunogenic fragment of the Matrix Protein, the H3 protein, the N2 protein or the N8 protein of the Canine influenzavirus. In some cases, the antibody that is used for detection of the CIRDC infection may be an antibody with affinity to an immunogenic fragment of the Glycoprotein, the Fusion protein, the Matrix protein or the SH protein of the Canine Pneumovirus. In some cases, the antibody that is used for detection of the CIRDC infection may be an antibody with affinity to an immunogenic fragment of the MAP protein or the CPS protein of Streptococcus equi. In some cases, the antibody that is used for detection of the CIRDC infection may be an antibody with affinity to an immunogenic fragment of the Cp protein of Canine bocavirus. In some cases, the antibody that is used for detection of the CIRDC infection may be an antibody with affinity to an immunogenic fragment of the E1 or the E2 protein of Canine hepacivirus.

In some cases, the method described herein calls for improved specificity of detection by the simultaneously identifying multiple antigens that may be produced by the pathogen upon infecting the animal. For example, the assay may employ methods for simultaneous detection of multiple virulence factors or immunogenic proteins of Bordetella bronchiseptica. In some cases, a combination of one or more of the proteins selected from Bordetella bronchiseptica filamentous hemagglutinin (FHA) protein or a part thereof, Bordetella bronchiseptica pertactin (PER) protein or a part thereof, Bordetella bronchiseptica fimbriae (FIM) protein or a part thereof, Bordetella bronchiseptica tracheal colonization factor or a part thereof, Bordetella bronchiseptica serum resistance factor (BrkA) or a part thereof, Bordetella bronchiseptica Bvg8 protein or a part thereof, Bordetella bronchiseptica O-antigen protein or a part thereof, Bordetella bronchiseptica adenylate cyclase-hemolysin or a part thereof, Bordetella bronchiseptica dermonecrotic toxin or a part thereof and Bordetella bronchiseptica tracheal cytotoxin or a part thereof, may be used as the subset of antigens for identifying the presence of the Bordetella bronchiseptica infection.

In some cases, one or more antibodies with specific affinity to one or more CIRDC pathogens or one or more immunogenic components thereof (e.g., Bordetella bronchiseptica antigens) may be used to detect the presence or absence of the pathogen in the sample obtained from the animal. The disclosure herein provides a solid support for the formation of multiple such antigen-antibody complexes. In one embodiment, the solid support comprises spatially distinct zones wherein each zone is pretreated with a unique antibody with affinity to a different immunogenic protein or virulence factor produced by the CIRDC pathogen. In one embodiment, the solid support described is an immunochromatographic assay strip. In one embodiment, the assay strip comprises a first zone pre-treated (coated) with a first antibody, wherein the first antibody is an antibody with affinity to a first protein of a first CIRDC pathogen or an immunogenic fragment thereof, a second zone pre-treated (coated) with a second antibody, wherein the second antibody is an antibody with affinity to a second protein of a second CIRDC pathogen or a fragment thereof that is different from the first antibody of the first zone, a third zone pre-treated (coated) with a third antibody wherein the third antibody is an antibody with affinity to a third protein of a third CIRDC antigen or a fragment thereof, that is different from the first antibody of the first zone and the second antibody of the second zone. In some cases, the first, second and the third pathogen may be the same CIRDC pathogen. In some cases, the first, second and the third pathogen may be different CIRDC pathogens.

In one embodiment, the method of detecting a CIRDC infection in an animal by measuring the presence of one or more of the CIRDC pathogens or one or more immunogenic fragments thereof, comprises the following steps: collecting a sample of one or more bodily fluids from the animal, wherein the one or more agents is present in the sample; applying the sample to a cassette unit, wherein the cassette unit is further capable of receiving a plurality of immunochromatographic assay strips; contacting the biological sample to a plurality of immunochromatographic assay strips; running a lateral flow assay along the immunochromatographic assay strip, wherein the running comprises allowing the sample to flow laterally along an assay strip, wherein the assay strip comprises at least one membrane-bound zone comprising at least one membrane-bound detector with affinity to the one or more immunogenic proteins produced by a CIRDC pathogen; allowing the formation of at least one label-antibody conjugate, wherein the conjugate is capable of generating a signal in one or more unique areas along the immunochromatographic assay strips that can be detected by a scanner; generating the signal; and detecting the signal, wherein the presence of the agent in the sample is determined by the presence or absence of the signal in one or more spatially distinct zones in the assay strip.

Provided herein are methods and systems for detecting a previous or current CIRDC infection in an animal that may have previously contracted CIRDC or that may have received a vaccine to one or more CIRDC pathogens (e.g., dog) by identifying one or more antibodies produced as a response to the CIRDC pathogen in an animal upon infection or vaccination, wherein the antibody is present in a sample selected from one or more bodily fluids of the animal. In one aspect, the methods also allow for evaluating the immunization status of the animal (e.g., dog) to one or more of the pathogens capable of causing CIRDC. The method described herein comprises the following steps: collecting a sample of one or more bodily fluids from an animal that may have previously contracted CIRDC or that may have been vaccinated against one or more CIRDC pathogens; applying the sample to a solid support comprising at least one antigen (e.g., a CIRDC pathogen or an immunogenic protein thereof) with affinity to at least one antibody that may be produced in response to the same in the animal from whom the sample is derived, allowing the formation of at least one labeled antigen-antibody conjugate, wherein the conjugate is capable of generating a signal; allowing the signal to generate to detectable levels; and detecting the signal, wherein the presence of the antibody in the sample is determined by the presence or absence of the signal.

In some cases, the method described herein calls for improved specificity of detection of a previous or current CIRDC infection in the biological sample by simultaneously identifying multiple antibodies that may be generated by the immune system of the animal (e.g., dog) upon concurrent infection with one or more pathogens that can cause CIRDC infection. In some cases, one or more CIRDC pathogens or an immunogenic fragment thereof may be used to form a complex with one or more of the antibodies in the sample obtained from the animal. In some cases, a combination of one or more of the antibodies generated against the Bordetella bronchiseptica filamentous hemagglutinin (FHA) protein, Bordetella bronchiseptica pertactin (PER) protein, Bordetella bronchiseptica fimbriae (FIM) protein, Bordetella bronchiseptica tracheal colonization factor, Bordetella bronchiseptica serum resistance factor (BrkA), Bordetella bronchiseptica Bvg8 protein, Bordetella bronchiseptica O-antigen protein, Bordetella bronchiseptica adenylate cyclase-hemolysin, Bordetella bronchiseptica dermonecrotic toxin and Bordetella bronchiseptica tracheal cytotoxin, may be used as the subset of antibodies for characterizing the presence of a previous or current infection and the subsequent immune response produced by the animal (i.e., dog) upon being infected by Bordetella bronchiseptica.

In some embodiments, the method of detecting a current or previous CIRDC infection caused by one or more CIRDC pathogens in an animal by measuring the presence of one or more antibodies generated against the one or more CIRDC pathogens or immunogenic fragments thereof by the animal comprises the following steps: collecting a sample of one or more bodily fluids from the animal, wherein the one or more antibodies is present in the sample; applying the sample to a cassette unit, and wherein the cassette unit is further capable of receiving a plurality of immunochromatographic assay strips; contacting the biological sample to a plurality of immunochromatographic assay strips; running a lateral flow assay along the immunochromatographic assay strip, wherein the running comprises allowing the sample to flow laterally along an assay strip, wherein the assay strip comprises at least one membrane-bound zone comprising at least one membrane-bound capture agent with affinity to the one or more Bordetella bronchiseptica agents; allowing the formation of at least one labeled labeled-antibody conjugate, wherein the conjugate is capable of generating a signal in one or more unique areas along the immunochromatographic assay strips that can be detected by a scanner; generating the signal; and detecting the signal, wherein the presence of the agent in the sample is determined by the presence or absence of the signal in one or more spatially distinct zones in the assay strip.

The disclosure herein provides methods of using a solid support for the detection of one or more antibodies generated by an infected animal against one or more CIRDC pathogens or immunogenic fragments thereof, wherein the solid support comprises spatially distinct zones wherein each zone comprises a unique antigen (immunogenic component of a CIRDC pathogen) with affinity to a different antibody that may be present in the biological sample from the animal. In one embodiment, the solid support described is an immunochromatographic assay strip. In one embodiment, the assay strip comprises a first zone pre-treated with a first antigen, a second zone pre-treated with a second antigen, wherein the second antigen is different from the first antigen of the first zone, a third zone pre-treated with a third antigen wherein the third antigen is different from the first antigen of the first zone and the second antigen of the second zone. In some cases, the first antigen may be the same as the second antigen. In some cases, the first antigen and the second antigen may be different.

In another embodiment, the method comprises attaching one or more of capture agents to the assay substrate. In one example, the method comprises attaching one or more of capture agents (e.g., capture antibodies specific for one or more Bordetella bronchiseptica antigens) to 1, 2, 3, 4, 5, 6, 7, 8, 9, 10, 11, 12, 14, 15, 16, 17, 18, 19, 20, 21, 23, 24, 25, 26, 28, 30, 31, 32, 34, 36, 39 or 40 spatially distinct locations (e.g., target reaction zones) on the solid support (e.g., nitrocellulose membrane). In one example, the method comprises attaching one or more of capture agents (e.g., capture antigens specific for one or more Bordetella bronchiseptica antibodies to 1, 2, 3, 4, 5, 6, 7, 8, 9, 10, 11, 12, 14, 15, 16, 17, 18, 19, 20, 21, 23, 24, 25, 26, 28, 30, 31, 32, 34, 36, 39 or 40 spatially distinct locations (e.g., target reaction zones) on the solid support (e.g., nitrocellulose membrane).

In one example, the method comprises attaching one or more anti-species antibodies (e.g., canine anti-IgA) to a distinct location (e.g., control zone) on the substrate (e.g., nitrocellulose assay strip). In one example, the method comprises attaching one or more control antibodies (e.g., antibody against an endogenous nasal protein) to a distinct location (e.g., control zone) on the substrate (e.g., nitrocellulose assay strip).

In one example, the method comprises attaching a known pre-measured concentration of probe-reporter pairs (e.g., antibody-HRP) to a unique known location (e.g., reference spot) onto the substrate of the assay strip.

In one example, the method comprises attaching one or more probe-reporter pairs (e.g., antibody-enzyme pair) specific for one or more target CIRDC pathogens or immunogenic fragments thereof to a distinct location (e.g., conjugate zone) on the substrate (e.g., nitrocellulose assay strip). In one example, the method comprises attaching one or more probe-reporter pairs (e.g., antigen-enzyme pair) specific for one or more target antibodies (e.g., an antibody generated against a CIRDC pathogen in an infected animal) to a distinct location (e.g., conjugate zone) on the substrate (e.g., nitrocellulose assay strip).

Methods of the present disclosure provide the detection of the formation of the capture agent-target analyte complex. In one aspect, following binding of the antigen from the sample, the capture antibody/target antigen complex is detected by any suitable method. In a different aspect, following binding of the antibody from the sample, the capture antigen-target antibody complex is detected by any suitable method. For example, the target-analyte-capture agent complex may be reacted with reporter-probe reagents (e.g., an enzyme-antibody conjugate) followed by detection of analyte (e.g., upon reaction with substrate).

The methods disclosed herein provide treating the assay membrane with an additional probe that has affinity to the target analyte (e.g., antibody to CIRDC antigen in an infected animal) of the target analyte-capture agent (e.g., antibody-antigen) complex. In some embodiments, this additional probe is an antibody with affinity to a Bordetella bronchiseptica pathogen or an immunogenic fragment thereof. In some embodiments, the target analyte-capture agent complex is detected when an indicator reagent, such as an enzyme conjugate, which may be bound to the capture agent or the target analyte is catalyzed by a detectable reaction. Optionally, a reporter reagent such as a signal generating compound may be applied to the target analyte-capture agent complex under conditions that allow formation of a detectable target analyte-capture agent-reporter reagent complex. Optionally, the capture agent may be labeled with a reporter reagent prior to the formation of a target analyte-capture agent-reporter complex.

Suitable methods for immobilizing peptides on solid phases include ionic, hydrophobic, covalent interactions and the like. Antibodies used in the device of the present disclosure may be immobilized on the solid support by any methodology known in the art, including, for example, covalently or non-covalently, directly or indirectly, attaching the antibodies to the solid support. Therefore, while these antibodies may be attached to the solid support by physical adsorption (i.e., without the use of chemical linkers), it is also true that these antibodies may be immobilized to the solid support by any chemical binding (e.g., with the use of chemical linkers) method readily known to one of skill in the art. In one aspect, the probe-reporter pairs or the capture agent may be adhered to the substrate using a protein carrier such as Bovine serum albumin or keyhole limpet hemocyanin.

Methods of the present disclosure include, but are not limited to those based on competition, direct reaction or sandwich-type assays, including, but not limited to ELISA, RIA, immuno-fluorescent assays (IFA), hemagglutination (HA), fluorescence polarization immunoassay (FPIA), and microtiter plate assays (e.g., any assay done in one or more wells of a microtiter plate). For example, an assay of the present disclosure includes a reversible flow chromatographic binding assay, which may be performed, for example, by using a SNAP® device. See U.S. Pat. No. 5,726,010.

In other embodiments, the method of the present disclosure includes a competition assay. In one embodiment, the method involves immobilizing capture molecules (e.g., antibodies against one or more Bordetella bronchiseptica antigens) at one or more of the target reaction zones and simultaneously contacting the immobilized capture antibodies with antigen from a sample and an antigen-reporter pair (e.g., an antigen-enzyme conjugate). In this embodiment, the amount of label detected at the target reaction zone may be inversely proportional to the amount of antigen in the sample.

In one embodiment, when a sample is applied to the first absorbent filter pad (e.g., sample pad), the sample laterally flows toward the second absorbent filter pad (at the distal end), washing over the one or more target reaction zones. The application of an improper sample (e.g., human sample) results in the probe-reporter pairs not binding to the capture agent present on the target reaction zone or with the analyte present in the sample and little or no binding with the antibodies present at the control zone. Conversely, when analyte is present in the sample, the free analyte binds to the probe in the probe-reporter pair and/or to the capture agent at the target reaction zone. The probe-reporter pair may also be bound by the antibodies at the control zone. With a lot of analyte present, the target reaction zone and the control zone may exhibit high binding with the probe-reporter pairs. Excess sample will be wicked into a second absorbent filter pad at the distal end of the assay strip.

In one embodiment, a saliva sample from an animal having respiratory symptoms including cough and nasal discharge is obtained. The sample is stabilized using a transport buffer and added to the sample input region of the assay strip. In some cases, one or more second anti-species antibodies may be added (e.g., Bordetella bronchiseptica). These second antibodies may be from a different species than are the solid phase capture antibodies. Third anti-species antibodies that specifically bind the second antibodies and that do not specifically bind the solid phase antibodies are added. The third antibodies may include an indicator reagent, such as an enzyme conjugate. Wash steps may be performed prior to each addition. A chromophore or enzyme substrate may be added and color may be allowed to develop. The color reaction may be stopped and the color may be quantified using, for example, a spectrophotometer, and/or the color may be subjectively assessed by the human eye.

### Antibodies

The present disclosure provides antibodies and antigen-binding fragments thereof that are raised against and that specifically bind all or part of one or more CIRDC pathogens or one or more immunogenic fragments thereof) present in the sample obtained from the animal (i.e., dog) and also includes compositions that include the antibodies and antigen-binding fragments thereof. When contacted to a sample obtained from an animal, these antibodies and antigen-binding fragments can specifically bind to a antigen (e.g., a CIRDC pathogen or an immunogenic fragment thereof). In one case, the pathogen is Bordetella bronchoseptica. For example, the antibodies and antigen-binding fragments can specifically bind to CIRDC antigens present in the sample but are not able to specifically bind any antigen from a pathogen that does not cause CIRDC and also may be present in the sample. The antibodies of the present disclosure are suitable for being used only to capture one or more CIRDC pathogens or one or more immunogenic fragments thereof, only to detect one or more CIRDC pathogens or one or more immunogenic fragments thereof, or more preferably, to both capture and detect one or more CIRDC pathogens or one or more immunogenic fragments thereof.

The antibodies of the present disclosure may belong to any antibody class, including for example, IgG, IgM, IgA, IgD and IgE, and may be prepared by any of a variety of techniques known to the skilled artisan. (See, e.g., Dean, Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68(1992); Harlow and Lane, Antibodies: A Laboratory Manual, Cold Spring Harbor Laboratory (1988); and Making and Using Antibodies: A Practical Handbook, Howard and Kaser, eds., CRC Press (2006), each one of which is incorporated herein by reference in its entirety.) It is also to be understood that the antibodies of the disclosure optionally may be polyclonal or monoclonal antibodies, single chain antibodies (scFv), chimeric antibodies, and fragments thereof. Monoclonal antibodies that are specific for the antigen peptide of interest may be obtained and purified, for example, by preparing cell lines that generate antibodies having the desired specificity to the antigen peptide of interest.

The immunogens used to stimulate the production of antibodies in animals can be purified proteins from disrupted organisms, synthetic peptides or recombinant proteins derived from specific protein sequences. Furthermore, specific protein sequences can be studied to identify any portion of a protein sequence that is more immunogenic and portions predicted to be less immunogenic such that a portion of a protein sequence thought to be the most immunogenic is made into a recombinant protein to use as an immunogen.

The antibodies of the disclosure also may be a single chain antibody (scFv), or an antigen binding fragment of an antibody. Antigen-binding fragments of antibodies are a portion of an intact antibody comprising the antigen binding site or variable region of an intact antibody, wherein the portion is free of the constant heavy chain domains of the Fe region of the intact antibody. Examples of antibody fragments include Fab, Fab′, Fab′-SH, F(ab′)2 and Fv fragments. In addition to production and purification from animals or mammalian cells, antibodies, antibody fragments, or non-antibody scaffolds can be selected based upon various in vitro technologies, including phage display, ribosomal display, or bacterial display.

Antibodies, including secondary antibodies, may be labeled with any type of label known in the art, including, for example, fluorescent, chemiluminescent, radioactive, enzymes, colloidal particles, radioisotopes and bioluminescent labels. In various embodiments of the disclosure, the one or more of the antibodies of the disclosure are labeled with an enzyme, a colloidal particle, a radionuclide or a fluorophore. The particulate label can be, for example, a colored latex particle, dye sol, or gold sol conjugated to an antibody.

### Solid Support

In one aspect, the device includes a solid support (e.g., assay strip), wherein one or more antibodies of the disclosure are immobilized on the solid support. The solid support may be, but is not limited to being, the inner, bottom surface of a well of a microtiter plate or a substrate that is included as part of a lateral flow device, for example. An exemplary microtiter plate is an Immulon IB 96-well plate (which is commercially available from Thermo Scientific of Milford, Mass.), but it is to be understood that the skilled artisan will recognize that a large variety of other microtiter plates that are not the Immulon IB 96-well plate allow for the immobilization of antibodies thereon, and therefore would be suitable for providing the solid support of the present disclosure.

The solid support may be a microtiter well, antibody-immobilizing portion of a SNAP® device, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). The membrane film can be glass fiber. These substrate materials may be used in suitable shapes, such as films, sheets, or plates, or they may be coated onto or bonded or laminated to appropriate inert carriers, such as paper, glass, plastic films, or fabrics. The strips can have a small relive pore size (5 μm) and a slow capillary flow rate (e.g., Merk Millipore HF 180 or HF135). The capillary flow rate can be between about 180 sec/cm and 135 (sec/cm). The capillary flow rate can be from about 100 sec/cm to about 200 sec/cm. Strip dimensions can be about 1 cm wide, and from about 5 to about 7 cm long.

It is also to be understood that the solid support substrate (e.g., assay strip) may be any suitable material for the immobilization of the antibodies of the disclosure. For example, the solid support (e.g., assay strip) substrate may be beads, particles, tubes, wells, probes, dipsticks, pipette tips, slides, fibers, membranes, papers, natural and modified celluloses, polyacrylamides, agaroses, glass, polypropylene, polyethylene, polystyrene, dextran, nylon, amylases, plastics, magnetite or any other suitable material readily known to one of skill in the art.

In one embodiment, the solid support can comprise a suitable material, e.g., a uniform-sized (10×500 mm) nitrocellulose membrane (Millipore™ XA3J072100). In one embodiment, the solid support can comprise a conjugate label pad (e.g., sample input pad) that is placed at one end of membrane. An absorption pad may be located at the opposite end of the membrane and may serve to draw the sample, e.g., saliva, along the membrane by capillary action. In one embodiment, a plastic backing may provide support for the adhesive layer and membrane, and the combination can be cut into individual test strips (e.g., 5×60 mm) and fitted into a plastic housing cassette. A sample application well may be positioned directly above and in fluid communication with the sample pad, and a detection window may be located above the nitrocellulose membrane.

The nitrocellulose layer may be a part of a lateral flow system, wherein a first absorbent filter pad is applied to the substrate, at proximal end of the assay strip, wherein the lateral flow system can wick a sample from one end to the other across the assay strip. In one embodiment of the disclosure the first absorbent filter pad, e.g., a sample pad, is capable of receiving a sample, filtering out any large particulate matter in the sample, and holding the sample so that it can slowly wick into the assay.

The assay strip further comprises a second absorbent filter pad located at the distal end of the assay strip. The second absorbent filter pad is capable of absorbing and holding the sample after it has wicked across the assay strip, preventing the sample from flowing in the opposite direction, and causing non-specific binding to occur.

In one embodiment of the disclosure, the assay strips, have a solid support substrate, which may be rigid or semi-rigid. This substrate may be made of a nitrocellulose layer; however, it is within the scope of the present disclosure that the layer is any material that is an insoluble matrix, including blotting material, capillaries, cellulose, silica, polystyrene, latex, or glass coated with a polymer.

In one embodiment, the assay strips of the present disclosure are disposable and are further capable of being releasably inserted into the cassette unit. In one embodiment, the assay strips of the present disclosure may be attached to the cassette unit. The cassette unit may be capable of receiving one or a plurality of assay strips, depending on the number of samples to be analyzed.

An exemplary lateral flow device is the lateral flow device that is described in U.S. Pat. No. 5,726,010, which is incorporated herein by reference in its entirety. In one embodiment, the device for performing a lateral flow assay may be a SNAP® device, which is commercially available from IDEXX Laboratories, Inc. of Westbrook, ME. However, it is to be understood that the skilled artisan will recognize that a large variety of other lateral flow devices that are not SNAP® devices or described by U.S. Pat. No. 5,726,010 allow for the immobilization of an antibody thereon, and therefore would be suitable for being used as the device of the present disclosure. These devices can include, for example, lateral flow devices that use colloidal gold technology.

### Probe-Reporter Pairs

In one embodiment, a conjugate is bound to the substrate of the assay strip. In another embodiment, the first absorbent filter pad (e.g., sample pad) is further capable of receiving a conjugate, comprising probe-reporter pairs, and the conjugate is applied to the first absorbent filter pad. In one example, the conjugate is comprised of assay reporter bound to a probe antibody; however, it is within the scope of the disclosure that the assay reporter may be associated with any molecule with an affinity to a target analyte, including molecules that can bind to antigens, proteins, nucleic acids, cells, sub-cellular organelles, and other biological molecules. Alternatively, the assay may be set up as a competitive design where the assay reporter is conjugated to a known amount of target analyte and captured via an antibody or other compound with a specific affinity for the target analyte.

In one embodiment, the probe-reporter pairs comprise a plurality of pairs of a probe bound to an assay reporter. In one embodiment, the probe is an antibody that is specific to a target analyte, which may be a Bordetella bronchiseptica antigen potentially present in the sample. In another embodiment, the probe is an antigen that is specific to a target analyte which may be a Bordetella bronchiseptica antibody that may be present in the sample obtained from the animal. In another example, the probe-reporter pair may comprise a known amount of the target analyte bound to the assay reporter. The probe-reporter pair may be dried down into the first absorbent filter pad (e.g., sample pad).

In one example, the assay reporter is an indicator reagent including a signal generating compound. Indicator reagents including signal generating compounds (labels) associated with the probe and may include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. In one embodiment, the assay reporter is a fluorescent semi-conducting nanocrystal also known as a quantum dot (including QDots®, available from Quantum Dot Corp., Hayward, Calif, and EviTag® Quantum Dots, available from Evident Technologies, Troy, N.Y.). The selection of a particular label is not critical, but it will be capable of producing a signal either by itself or in conjunction with one or more additional substances. Additionally, within other alternate embodiments of the present disclosure, the conjugate probe-reporter pair may comprise standard assay reporters (e.g., labeled colloidal gold, latex beads, lanthanide-doped ceramic nanoparticles) that are colloidally stabilized, highly sensitive, small diameter particles with high surface area/volume ratios capable of attachment of biological ligands, and bound to the probe (e.g., antibody or antigen) to form the probe-reporter pair.

In one embodiment, the assay reporter can detect the presence or amounts of a biological or chemical moiety; the structure, composition, and conformation of a biological moiety; the localization of a biological or chemical moiety in an environment; interactions of biological or chemical moieties; alterations in structures of biological or chemical compounds; and alterations in biological or chemical processes. In one embodiment, the assay reporter has a characteristic spectral emission, which is tunable to a desired energy by selection of the particle size of the quantum dot and an affinity for a target analyte. In one embodiment, the location and nature of the association can be detected by monitoring the emission of the assay reporter.

In one embodiment, the assay reporter has a range of excitation wavelength that is broad and allows the simultaneous excitation of all assay reporters in a system with a single light source, and is resistant to degradation or photobleaching over time.

In one embodiment, one or more probe-reporter reagents may be mixed with a sample from an animal (e.g., dog) prior to application to a device herein. In another embodiment, the probe-reporter pairs described above or the capture agent (e.g., antibody specific for a given target Canine hepacivirus antigen) may be directly or indirectly attached to a solid support or a substrate. In one example, capture antigens specific for one or more Canine bocavirus antibodies may be directly or indirectly attached to the solid support. Suitable methods for immobilizing peptides on solid phases include ionic, hydrophobic, covalent interactions and the like. Antibodies used in the device of the disclosure may be immobilized on the solid support by any methodology known in the art, including, for example, covalently or non-covalently, directly or indirectly, attaching the antibodies to the solid support. Therefore, while these antibodies may be attached to the solid support by physical adsorption (e.g., without the use of chemical linkers), it is also true that these antibodies may be immobilized to the solid support by any chemical binding (e.g., with the use of chemical linkers) method readily known to one of skill in the art. In one aspect, the probe-reporter pairs or the capture agent may be adhered to the substrate using a protein carrier such as Bovine serum albumin or keyhole limpet hemocyanin.

The assay strip may further comprise up to 1, 2, 3, 4, 5, 6, 7, 8, 9, 10, 11, 12, 14, 15, 16, 17, 18, 19, 20, 21, 23, 24, 25, 26, 28, 30, 31, 32, 34, 36, 39 or 40 target reaction zones comprising substrate-bound capture agents in spatially distinct zones on the assay strip. In one embodiment, the target reaction zones are applied and dried onto the substrate of the assay strip. “Capture agent” or ‘capture molecule’ refers to any compound that is specific for the ‘antigen’ or ‘antibody’ of interest. The “capture agent” or the “capture molecule” may be a capture antigen or a capture antibody. In one embodiment, the target reaction zone may comprise capture agents (e.g., antibody) bound to the assay strip, which are capable of “capturing” one or more immunogenic proteins of one or more CIRDC pathogens (e.g., Canine hepacivirus antigens present in the sample), via antigen-antibody affinity binding, when the sample flows past the capture line. In one embodiment, the target reaction zone may comprise capture agents (e.g., one or more immunogenic fragments of one or more CIRDC pathogens) bound to the assay strip, which are capable of “capturing” one or more antibodies generated by the animal against one or more CIRDC pathogens upon infection (e.g., Canine hepacivirus antigens present in the sample), via antigen-antibody affinity binding, when the sample flows past the capture line. In one embodiment, 1, 2, 3, 4, 5, 6, 7, 8, 9, 10, 11, 12, 13, 14 or 15 control zones may be present in the device. In another embodiment, each control zone may comprise a different capture molecule. In another embodiment, the capture molecules in at least two of the control zones are the same. In one embodiment, the assay strip may comprise a first target reaction zone comprising a first substrate-bound capture antigen of Bordetella bronchiseptica, a second target reaction zone with a second substrate-bound capture antigen of Bordetella bronchiseptica that is different from the first capture antigen of the first zone, a third target reaction zone with a third substrate-bound capture antigen of Bordetella bronchiseptica that is different from the first antigen of the first zone and the second antigen of the second zone. In another embodiment, the assay strip may comprise a first target reaction zone comprising a first substrate-bound capture antibody of Bordetella bronchiseptica, a second target reaction zone with a second substrate-bound capture antibody of Bordetella bronchiseptica that is different from the first antibody of the first zone, a third target reaction zone with a third substrate-bound capture antibody of Bordetella bronchiseptica that is different from the first antibody of the first zone and the second antibody of the second zone.

In one embodiment, the assay strip may comprise optionally a first target reaction zone comprising one or more substrate-bound capture antigens of Bordetella bronchiseptica, optionally a second target reaction zone with one or more substrate-bound capture antigens of Canine parainfluenza virus, optionally a third target reaction zone with one or more substrate-bound capture antigens of Canine coronavirus, optionally a fourth target reaction zone with one or more substrate-bound capture antigens of Canine adenovirus type-2, optionally a fifth target reaction zone with one or more substrate-bound capture antigens of Canine herpesvirus, optionally a sixth target reaction zone with one or more substrate-bound capture antigens of Canine distemper virus, optionally a seventh target reaction zone with one or more substrate-bound capture antigens of Canine Influenzavirus, optionally an eighth target reaction zone one or more substrate-bound capture antigens of Canine Pneumovirus, optionally a ninth target reaction zone with one or more substrate-bound capture antigens of Mycoplasma cynos, optionally a tenth target reaction zone with one or more substrate-bound capture antigens of Streptococcus equi, optionally an eleventh target reaction zone with one or more substrate-bound capture antigens of Canine bocavirus and optionally a twelfth target reaction zone with one or more substrate-bound capture antigens of Canine hepacivirus.

In one embodiment, the assay strip may comprise optionally at least one target reaction zone comprising one or more substrate-bound capture antigens of Bordetella bronchiseptica, optionally at least one target reaction zone with one or more substrate-bound capture antigens of Canine parainfluenza virus, optionally at least one target reaction zone with one or more substrate-bound capture antigens of Canine coronavirus, optionally at least one target reaction zone with one or more substrate-bound capture antigens of Canine adenovirus type-2, optionally at least one target reaction zone with one or more substrate-bound capture antigens of Canine herpesvirus, optionally at least one target reaction zone with one or more substrate-bound capture antigens of Canine distemper virus, optionally at least one target reaction zone with one or more substrate-bound capture antigens of Canine Influenzavirus, optionally at least one target reaction zone one or more substrate-bound capture antigens of Canine Pneumovirus, optionally at least one target reaction zone with one or more substrate-bound capture antigens of Mycoplasma cynos, optionally at least one target reaction zone with one or more substrate-bound capture antigens of Streptococcus equi, optionally at least one target reaction zone with one or more substrate-bound capture antigens of Canine bocavirus and optionally at least one target reaction zone with one or more substrate-bound capture antigens of Canine hepacivirus.

In another embodiment, the assay strip may comprise optionally a first target reaction zone comprising one or more substrate-bound capture antibodies with affinity to one or more immunogenic fragments of Bordetella bronchiseptica, optionally a second target reaction zone comprising one or more substrate-bound capture antibodies with affinity to one or more immunogenic fragments of Canine parainfluenza virus, optionally a third target reaction zone comprising one or more substrate-bound capture antibodies with affinity to one or more immunogenic fragments of Canine coronavirus, optionally a fourth target reaction zone comprising one or more substrate-bound capture antibodies with affinity to one or more immunogenic fragments of Canine adenovirus type-2, optionally a fifth target reaction zone comprising one or more substrate-bound capture antibodies with affinity to one or more immunogenic fragments of Canine herpesvirus, optionally a sixth target reaction zone comprising one or more substrate-bound capture antibodies with affinity to one or more immunogenic fragments of Canine distemper virus, optionally a seventh target reaction zone comprising one or more substrate-bound capture antibodies with affinity to one or more immunogenic fragments of Canine Influenzavirus, optionally an eighth target reaction zone comprising one or more substrate-bound capture antibodies with affinity to one or more immunogenic fragments of Canine Pneumovirus, optionally a ninth target reaction zone comprising one or more substrate-bound capture antibodies with affinity to one or more immunogenic fragments of Mycoplasma cynos, optionally a tenth target reaction zone comprising one or more substrate-bound capture antibodies with affinity to one or more immunogenic fragments of Streptococcus equi, optionally an eleventh target reaction zone comprising one or more substrate-bound capture antibodies with affinity to one or more immunogenic fragments of Canine bocavirus and optionally a twelfth target reaction zone comprising one or more substrate-bound capture antibodies with affinity to one or more immunogenic fragments of Canine hepacivirus.

In another embodiment, the assay strip may comprise optionally at least one target reaction zone comprising one or more substrate-bound capture antibodies with affinity to one or more immunogenic fragments of Bordetella bronchiseptica, optionally at least one target reaction zone comprising one or more substrate-bound capture antibodies with affinity to one or more immunogenic fragments of Canine parainfluenza virus, optionally at least one target reaction zone comprising one or more substrate-bound capture antibodies with affinity to one or more immunogenic fragments of Canine coronavirus, optionally at least one target reaction zone comprising one or more substrate-bound capture antibodies with affinity to one or more immunogenic fragments of Canine adenovirus type-2, optionally at least one target reaction zone comprising one or more substrate-bound capture antibodies with affinity to one or more immunogenic fragments of Canine herpesvirus, optionally at least one target reaction zone comprising one or more substrate-bound capture antibodies with affinity to one or more immunogenic fragments of Canine distemper virus, optionally at least one target reaction zone comprising one or more substrate-bound capture antibodies with affinity to one or more immunogenic fragments of Canine Influenzavirus, optionally at least one target reaction zone comprising one or more substrate-bound capture antibodies with affinity to one or more immunogenic fragments of Canine Pneumovirus, optionally at least one target reaction zone comprising one or more substrate-bound capture antibodies with affinity to one or more immunogenic fragments of Mycoplasma cynos, optionally at least one target reaction zone comprising one or more substrate-bound capture antibodies with affinity to one or more immunogenic fragments of Streptococcus equi, optionally at least one target reaction zone comprising one or more substrate-bound capture antibodies with affinity to one or more immunogenic fragments of Canine bocavirus and optionally at least one target reaction zone comprising one or more substrate-bound capture antibodies with affinity to one or more immunogenic fragments of Canine hepacivirus.

Provided here in Table I are a partial list of immunogenic parts of various CRDIC pathogens. The various antigens listed here can be used as target analytes or capture reagents in the assay. Antibodies with affinity to one or more of these immunogenic components may be used as target analytes or capture agents in the assay.

TABLE 1
Immunogenic components of CIRDC pathogens that may be used
as target analytes or capture agents in the assay

| Organism | Antigen/Antibody Target analytes or capture agents |
|---|---|
| Bordetella | Filamentous Hemagglutinin protein(FHA), |
| brochiseptica | Pertactin protein (PRN), branched chain |
| | fatty acid Bordetella Colonization Factor |
| | A (BcfA) protein |
| Canine | Hemagglutinin-nueraminidase protein, |
| parainfluenza | Fusion protein, Matrix protein |
| virus | |
| Canine respiratory | Spike Glycoprotein trimer protein, Membrane |
| coronavirus | protein, Nucleoprotein, or Envelope small |
| | membrane pentamer protein |
| Canine adenovirus | Capsid protein, Fiber protein, Penton protein, |
| type-2 | Hexon protein |
| Canine herpesvirus | Envelope and Major capsid proteins |
| Canine distemper | Hemagglutinin (H), Nucleocapsid, and the |
| virus | Fusion Protein (F) |
| Canine Influenza | Matrix protein, Nucleocapsid, H3 protein, N2 |
| virus | protein, and N8 protein |
| Canine Pneumo | Glycoprotein, Fusion protein, Matrix protein, |
| virus | SH protein |
| Mycoplasma cynos | Major Lipoprotein, Hemagglutinin A, Sialidase |
| Streptococcus equi | MAP protein, CPS protein |
| Canine bocavirus | Cp protein |
| Canine hepacivirus | El protein and E2 protein |

In other embodiments of the method of the present disclosure, a competition assay is performed. In one embodiment, the capture molecules may be a known concentration of the specific target analyte (e.g., antigen), further capable of capturing reporter-probe pairs that are not bound to analyte present in the sample. Accordingly, the fluorescence emitted at the target reaction zone will decline with increasing amounts of analyte present in the sample. In one embodiment of a competition assay, capture antigens are immobilized at the target reaction zone and are contacted simultaneously with antigen from a sample and antigen-reporter pair (e.g., an antigen-enzyme conjugate). The amount of assay reporter detected at the target reaction zone is inversely proportional to the amount of antigen in the sample. For example, the assay strip may comprise a target reaction zone comprising a first capture antigen of Bordetella bronchiseptica, wherein the capture antigen can capture antibody-reporter pairs (e.g., probe-reporter pairs) that are not bound to antigen present in the sample.

It is further contemplated that the assay strip may alternatively use traditional assay reporters for detecting analytes including radioactive markers; fluorescent molecules as tags including mono or polyclonal antibodies labeled with a fluorescent tag (e.g., fluorescein, propidium iodide, Hoechst dyes, ethidium bromide, methyl coumarin, or Texas red) and directed at a particular target; and secondary antibodies tagged with a fluorescent marker and directed to the primary antibodies to visualize the target.

The device of the present disclosure may also include various binding reagents immobilized at locations distinct from the target reaction zone. For example, the assay strip comprises a control zone wherein an immunoreagent (an antibody, antigen or polypeptide) that recognizes a species-specific (e.g., Canine bocavirus) antibody portion of an antibody-reporter or antigen-reporter reagent, or an enzyme portion of an enzyme-labeled probe-reporter reagent, can be included as a positive control to assess the viability of the reagents within the device. For example, a positive control may be an anti-horseradish peroxidase antibody that has been raised in, for example, goat or mouse. In one embodiment, the assay strip comprises a ‘control zone’ with a known concentration of commercially available anti-species antibody. Anti-species antibodies are capable of binding with the reporter-probe pair whether target analytes are present or not and serve to indicate whether the assay is functioning properly, and may be proportional to the amount of reporter-probe pairs bound to the target analyte in the sample, providing another level of sensitivity to the assay. Additionally, a reagent, e.g., an antibody, isolated from a non-immune member of the species from which the antibody portion of the antigen-antibody complex was derived can be included as a negative control to assess the specificity of immunocomplex (e.g., antigen-antibody complex) formation.

In multiple embodiments of the disclosure, the assay strips also include a reference spot, comprising a known pre-measured concentration of probe-reporter pairs spotted and dried down to a unique known location, onto the substrate of the assay strip. The reference spot may be any shape or size, including a circular spot or a line. The known pre-measured concentration of probe-reporter pairs at the reference spot will produce a signal reading, which serves as a control, allowing the reader to detect the quantity of signal produced by the reference spot. The reference spot is further capable of providing calibration for the instrument and comparing assay test results to this control. Use of the reference spot further allows automatic calibration of the diagnostic assay reader in the field.

The device also may optionally include a liquid reagent that transports, or otherwise facilitates removal of (such as when the device includes a microtiter plate, for example), unbound material (e.g., unreacted portions of the animal sample, such as, for example, unreacted portions of nasal extract, and unbound capture reagent) away from the reaction zone in the solid support (e.g., assay strip). The liquid reagent may be a wash reagent and serve only to remove unbound material from the reaction zone, or it may include a detector reagent and serve to both remove unbound material and facilitate antigen detection. For example, in the case of an antibody-HRP or an antigen-HRP (probe-reporter conjugate), the detector reagent includes a substrate that produces a detectable signal upon reaction with the probe-reporter conjugate at the target reaction zone on the solid support. Alternatively, in the case of a probe-reporter conjugate wherein the assay reporter is a radioactive, fluorescent, or light-absorbing molecule, the liquid reagent may act as a wash solution facilitating detection of complex formation at the target reaction zone by washing away unbound labeled reagent. The liquid reagent may further include a limited quantity of an “inhibitor”, e.g., a substance that blocks the development of the detectable signal. A limited quantity is defined as being an amount of inhibitor sufficient to block signal development until most or all excess, unbound material is transported away from the target reaction zone, at which time detectable signal is produced.

In addition to being designed to specifically binding and isolating Bordetella bronchiseptica antigens from the sample of an animal, the device of the disclosure optionally may be designed to allow one or more other diagnostic tests to be performed. For example, the solid support may also include reagents for the detection of one or more parasites, one or more viruses, one or more fungi, or one or more bacteria. The reagents for the detection of one or more non-worm parasites, one or more viruses, one or more fungi, or one or more bacteria may be, for example, one or more antibodies or one or more antigens recognized by antibodies specific for one or more non-worm parasites, one or more viruses, one or more fungi, or one or more bacteria.

### Device Unit

The device unit can be a cassette unit. The cassette unit holds the assay strips for application of the sample and for scanning of the assay strips inside the reader. The cassette unit may be a disposable single-use device or re-loadable so that assay strips may be replaced after use. In some embodiments, the cassette unit is circular with a hub as an axial pivot point, and with attached assay strips extending radially from the hub in a spoke-like configuration. In some embodiments, the cassette unit is oval, rectangular, square or amorphous.

In this embodiment, the spokes outwardly connect from a central sample port at hub. The cassette unit design is such that each assay strip is mounted in a fashion that allows each assay to flow laterally from the hub, outward toward the perimeter of the cassette unit. In one embodiment of the disclosure, each cassette unit holds a plurality of fixedly-mounted single-use assay strips. The cassette units are removable from the diagnostic assay reader and may be archived or disposed of. In another embodiment of the disclosure, the cassette unit is reusable and can receive new single-use assay strips after processed ones are removed.

The cassette unit may be fabricated of any lightweight rigid material, such as metal, polystyrene, polycarbonate, or similar durable plastic. Some method of attaching assay strips to the cassette may be provided. In one embodiment, appropriately-sized shallow slots may be formed in the cassette unit surface, into which assay strips may be inserted. In another embodiment, the assay strips may be manufactured with an adhesive backing on the bottom surface, allowing them to be stuck onto the cassette unit disc.

If the cassette unit is to be used with a reader unit that provides for an excitation source positioned beneath the assay strip, then the cassette unit must be transparent to the excitation energy, at least directly under the assay strip. For example, the cassette unit material under the slots might be clear plastic. Alternatively, the entire cassette unit body may be clear plastic. In addition, the excitation source may be positioned above the assay strips with a control for turning it off prior to reading the emission.

In one embodiment, the cassette unit is designed to receive the sample from a pipette in which the sample is taken or stored. The pipette mates to the sample port, and the water is injected. The sample is subdivided or split simultaneously upon application by a sample distributor, such that pre-determined volumes are selectively directed to a plurality of assay strips. The assay strips may each be for a single specific analyte or some redundancy may be employed. In this embodiment of the disclosure, a sample distributor comprises a cone configured with a plurality of channels down the outer surface of the cone to distribute a portion of the sample into each assay strip. The distributor may channel an equal amount of the sample to each of the radiating assay strips, or it may, where warranted, divide and direct unequal amounts of sample to specific assay strips.

Cassette unit configurations other than the disc with spokes may also be used. For example, a parallel slot configuration may be desirable in some applications. Configurations of any alternative cassette would have to match the configuration of the reader with which it is used. In one example, the device may comprise a single assay substrate with an array of target reaction zones wherein each target reaction zone may comprise one or more substrate-bound capture molecules. In one configuration, the array of target reaction zones may comprise both antigens and antibodies as substrate-bound capture molecules in order to make the assay more robust. In one example, each zone may comprise a unique composition of capture molecules. In one example, the target reaction zones may comprise a non-unique combination of capture molecules. In one example, the device may comprise a single assay substrate with an array of target reaction zones wherein each zone comprises at least one substrate-bound capture antigen each of which can identify one or more antibodies generated in the animal upon infection by one or more CIRDC pathogens. In one example, the device can comprise at least a single assay substrate comprising an array of target reaction zones each zone comprising substrate-bound capture antibodies each of which can identify at least one or more immunogenic components of at least one CIRDC pathogen.

The array may be configured as a two-dimensional arrangement of dots. In one example, the array of target reaction zones may be a series of stripes arranged parallel to each other, wherein each target reaction zone comprises one or more substrate-bound capture molecules (e.g., antibodies that can identify one of more immunogenic components of one or more CIRDC pathogens). In one configuration, the array of capture molecules may be present distal to a sample input zone and proximal to a wicking pad that absorbs the sample and pulls it toward the distal end of the assay substrate. In one configuration, the device may comprise a filter pad that can filter the sample of contaminants before reaching the target reaction zones. configuration might comprise an array of dots (zones) wherein each dot comprises at least one antigen with affinity to at least one antibody that is generated by the animal in response to a CIRDC antigen (e.g. Canine bocavirus). The filter can comprise cellulose fibers or a woven mesh. The filter can be manufactured by the compression of fibers of cellulose, glass, or plastic (such as polyester, polypropylene, or polyethylene) into thin mats. The sample could be flowed into a manifold trough for distribution to the assay strips, or samples could be applied to the proximal pad directly from a dropper or other device.

In one example, the substrate may be a nitrocellulose membrane strip. In one example, the membrane strip may up to 0.2 inches wide, 0.5 inches wide, I inch wide, 2 inches, 3 inches, 4 inches or 5 up to 5 inches wide. The strip may be I inch long, 2 inches long, 3 inches long, 4 inches long, 5 inches long or up to 6 inches long.

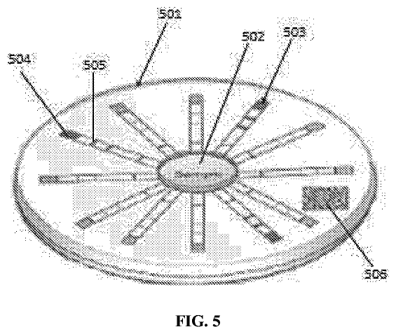

In some aspects, as can be seen in FIG. 5, the device 501 can comprise one or more strips 503, each comprising discrete sections, 504, 505. One discrete section can comprise a control analyte binding agent 504. Another discrete section can comprise a target analyte binding agent 505. The device can have a central sample input 502. The central sample input can comprise a filter. The device can comprise a code 506, (e.g., QR code) to identify the target analytes of the device to a computer program.

### Assay Reader

In one embodiment, the diagnostic assay reader is a hand-held unit. The diagnostic assay reader may be capable of reading assays, recording results, and archiving multiple results for later retrieval and analysis. In one embodiment, the assay reader may be configured to accept a cassette unit, containing a plurality of assay strips, via a slot.

In yet another embodiment, the diagnostic assay reader comprises a portable, closeable instrument case configuration. In one embodiment, the configuration includes one or a plurality of features including: protective case, battery storage, accommodation for larger batteries, storage for additional cassette units and storage for additional array strips.

In at least one embodiment, the diagnostic assay reader can include a screen display and memory capable of receiving user-entered data (e.g., location site name, latitude, longitude from separate GPS, weather conditions, date and time) using an interface similar to a Personal Data Assistant or a mobile phone or a tablet. User data may be entered via a touch screen or a keypad.

In one embodiment, the diagnostic assay reader may provide an excitation source and an emission receptor to extract information from the assay strips, and wherein the excitation source is a source that provides the appropriate range of excitation based on the type of assay reporters used in the assay. In one embodiment, the excitation source may be an ultraviolet light source, positioned in the reader case below the assay strip being processed. In one embodiment, the assay reader may comprise a photodiode detector array emission receptor, which senses the fluorescent energy radiated by the conjugates on the strip in the predetermined wavelength and maps the intensity distribution along the length and across the width of the strip. The intensity distribution is then stored or displayed on both.

In one embodiment, the system comprises a reader matched to a circular cassette with radial alignment of assay strips. In one embodiment, the strip may be aligned under the detector array and reading taken by activating the excitation source and emission receptor. Once the reading is complete, the cassette unit may be rotated until the next strip aligns with the detector and that assay strip may be read.

### Reading the Assay Strips

When a user is ready to read the assay strips, a cassette unit, filled with one or a plurality of assay strips, may be inserted into a slot in the diagnostic assay reader. In another embodiment, the diagnostic assay reader may first receive a designated signal from the reference spot as an internal diagnostic control. In one embodiment, reference spots may be integrated into the assay strip or cassette unit for each assay in order to function as an internal instrument check and as a signal comparator or reference to which the assay signal will be compared. In one embodiment, the reference spot may provide a relative standard (subject to the same ambient conditions) as the test signal.

In one embodiment, once the reference spot is read, the signal may be stored in the electronic data storage unit and compared to the signal from the control zone and target reaction zone on the assay strip. In one embodiment, during assay development for specific analytes, signal/concentration data may be determined for each assay control zone and target reaction zone and programmed into the electronic data storage unit for comparison to the reference spot. In one embodiment, the diagnostic assay reader may detect the signal emitted from the control zone and the target reaction zone, these signal readings may be directly proportional to the concentration of analyte in the test sample.

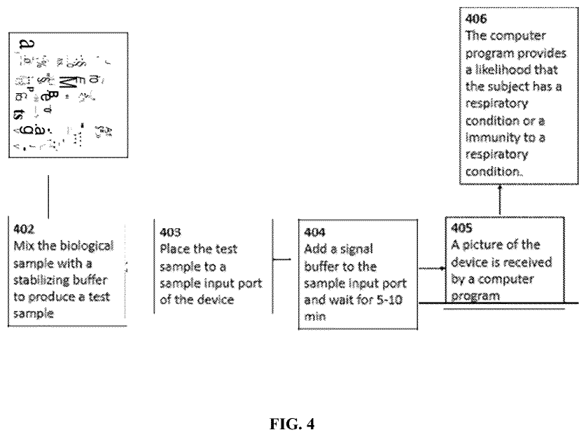

In one aspect, as can be seen in FIG. 4, a biological sample from an anterior nasal, oral, or conjunctiva! area of a subject is obtained 401 and mixed with a stabilizing buffer to produce a test sample 402. The test sample is then placed into the sample input port of a device as described herein 403. A signal buffer is then placed into the sample input port and left for 5-10 minutes to saturate the strips 404. A picture of the device is then received by a computer program 405. The computer program then provides a likelihood that the subject has a respiratory condition or has an immunity to a respiratory condition 406.

In another embodiment of the disclosure, data may be reported as brightness or as a color change. In one embodiment, the data may be collected and interpreted based on the function of horizontal distance along the test strip, or the distance of migration of the probe-reporter along the assay strip, using techniques and analysis like gel electrophoresis. In another embodiment, the probe-reporter pairs would move along the strip to different distances depending on whether or not they were bound to the target analyte. The proportion of the intensity of the assay reporter signal between the two lines would be an indirect measure of analyte in the sample.

### Data Analysis

In one embodiment, assay data may then be generated as a binary profile, indicating in binary terms whether the target analyte is present in the sample, after allowing the instrument to compare the signal intensities of the target reaction zone, control zone, and reference spot, in the determination of analyte concentration.

In another embodiment, the assay data may be generated as an emission intensity profile, allowing the instrument to compare the signal intensities of the target reaction zone, control zone, and reference spot, in the determination of analyte concentration. In at least one embodiment of the disclosure, based on these intensities, an algorithm, such as a direct ratio, or a slope determination which varies inversely with sample concentration, is used to determine the analyte concentration present in the sample.

In one embodiment, the algorithm may be used to generate a quantitative or semi-quantitative result (for example the detection of a particular analyte displayed on the readout in parts per million or billion). Further, this data point may be stored such that the user inputs unique alphanumeric identifiers associated with the data point (such as location name, global positioning system coordinates, or other unique identifiers). The electronic data storage unit may allow the user to recall the data on screen display and to upload the data via wired or wireless connection to a personal computer for the creation of a database.

In one embodiment, several signal comparison algorithms may be executed during the operation of the diagnostic assay reader. One such algorithm may be a signal comparison between signals emitted by the sample and a pre-applied control reference spot for each assay that is integrated into the cassette. The reference spot may provide an emitted light signal at a unique address (location) on the strip. This address may be programmed into the diagnostic assay reader.

Before scanning the length of an assay strip, the diagnostic assay reader may first move to the reference spot to receive a start signal. The diagnostic assay reader may receive a positive threshold signal value from the reference spot. If this signal is not received, or is below an assigned threshold value, the diagnostic assay reader may not continue with the reading of the assay strip. In one embodiment, entering such an error mode may cause a visual or audible signal to be emitted to the operator, and/or cause one or more instructions to the reader to be displayed on the readout display. Similarly, if the control signal is equal to or above the threshold value, the reader may allow the user to continue with the assay procedure.

The reference spot signal may then be compared to one or more assay signals using a separate algorithm. This second algorithm may be used to compare signal levels from at least two separate locations on the assay strip and also to the control reference spot signal. An emission intensity profile is then generated. This may allow the instrument to compare the signal intensities of the target reaction zone, control zone, and reference spot in the determination of sample concentration.

A direct ratio of signal intensity can be used to determine analyte levels. For example, the signal ratio of one target reaction zone:control zone in a negative sample is 100:2 (=50); a medium positive would be 50:50, (=1); and a high positive would be 2:100 (=0.02). In one embodiment, the two zone signals may also be summed, and this sum divided by the signal from the reference spot. This normalizes the signal so direct comparison from sample-to-sample can be made. Another algorithm possibility is a slope determination, based on the sample and control zone peaks as the points that determine the zone. The resulting line slope will vary inversely with sample concentration.

In at least one embodiment of the disclosure, the assay data (binary or intensity profile as per distance) may determine a value of analyte concentration and may be presented on a screen display for immediate use. The intensity profile and concentration values may then be stored in non-volatile memory of the electronic data storage unit along with any test sample descriptive information entered by the user through a key pad or screen display. The display can be a user device. The user device can be a mobile device (e.g., smartphone, tablet, etc.), a computer (e.g., laptop computer, desktop computer, etc.), and/or a wearable device (e.g., smartwatches, etc.). The user device may be a network device capable of connecting to a network such as a local area network (LAN), wide area network (WAN) such as the Internet, intranet, extranet, a telecommunication network, a data network, and/or any other type of network. The user device can be used to access a computer program capable of analyzing a digital image of the device as described herein. The user device can have a code (e.g. QR code) by which the computer program can identify the control values that the device is to be compared to. The QR code can also be used encode test information and interpretation criteria on the device. The computer program can relay to the user device the results of the analysis. The computer program can save the results of the analysis in a user account. The computer program can save multiple results of multiple analyses in a user account. The computer program can track and graphically display the results of the analyses over time to aid in monitoring the progression of a condition over time. The computer program can be accessed through an application configured for a smartphone (e.g. an app). The computer program can be accessed through a website configured for display on a desktop or smartphone.

The foregoing description of preferred embodiments of the present disclosure has been provided for the purposes of illustration and description. It is not intended to be exhaustive or to limit the disclosure to the precise forms disclosed.

### EXAMPLES

The following examples are included for illustrative purposes only and are not intended to limit the scope of the disclosure.

### Example I: Evaluating the Bordetella bronchiseptica Immunization Status of an Animal

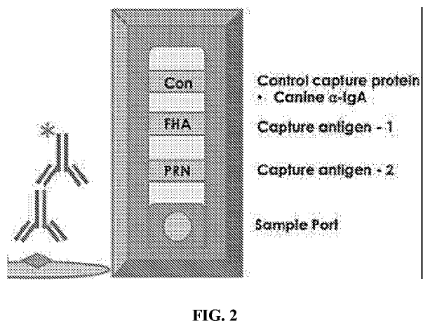

A nasal swab sample is obtained from a dog by rotating the swab for a few seconds. The sample is placed in a transfer buffer containing protease inhibitor. The sample is added dropwise to the device on a sample pad adjacent to the assay strip. The swab is then placed in the collection tube which contains about 3 mL of media. An aliquot of the sample (e.g., 500 μl-1 mL) is then placed in the sample port of the device. The assay strip comprises Bordetella bronchiseptica specific antigens pertactin and FHA in two distinct target reaction zones as shown in FIG. 2 and are attached to the substrate of the assay strip. The sample filters down through the sample pad and then through a conjugate label pad containing a conjugate label, e.g., Protein A conjugated with colloidal gold. The gold particles serve as an indicator dye. The conjugate label binds to Bordetella bronchiseptica specific anti-Per and anti-FHA IgG antibodies in the sample to form complexes, and the complexes then migrate along the membrane or detection strip. The complexes of Bordetella bronchiseptica anti-Per and anti-FHA IgG antibodies, if present, bind to the Bordetella bronchiseptica per and FHA antigens that are immobilized in a discrete target reaction zone location on the membrane. Formation of this gold-labeled Protein A antibody complex with its Bordetella bronchiseptica antigen results in a detectable colored line, indicating a positive result that Bordetella bronchiseptica specific antibodies are present in the sample. Formation of a colored line at the control zone indicates that the appropriate sample has been added and the assay has worked. The time for this test is about 5-10 minutes, and under 20 minutes.

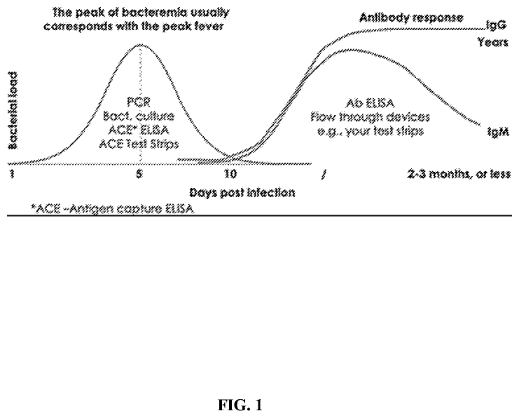

The results can be used to evaluate the Bordetella bronchiseptica immunization status of an animal as shown in FIG. 1. For example, if the sample tests positive, and the sample came from an animal that has received a Bordetella bronchiseptica vaccine, then the positive result will indicate that the appropriate immune response was elicited from that animal, particularly if there are no other indications or symptoms of Bordetella bronchiseptica in the animal. If the sample tests negative, and the sample came from an animal that has received a Bordetella bronchiseptica vaccine, then the negative result will indicate that the animal has not been properly immunized.

### Example 2: Evaluating the Presence of Bordetella bronchiseptica Infection in an Animal

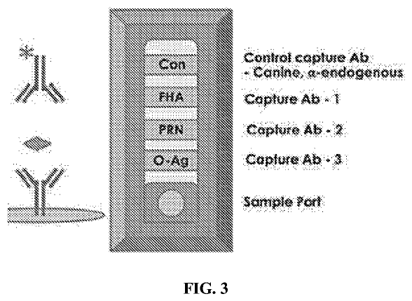

A saliva sample is obtained from a dog having symptoms of respiratory disease of unknown etiology. The sample is stabilized using a transport buffer containing anti-mycotic components as well as other inhibitors of protein degradation. The sample is then added to the sample pad region of the assay strip. The assay strip comprises antibodies specific to Bordetella bronchiseptica pertactin and FHA in two distinct target reaction zones as shown in FIG. 3 and are attached to the substrate of the assay strip. In the next step HRP-linked antibodies such as anti-Per-HRP and anti-FHA-HRP are added to specifically bind to one or more of the Pertactin and FHA antigens that may be present in the sample. The attached antibodies (anti-Per and anti-FHA) bind to the antigens (Per and FHA respectively) that may also be bound to the enzyme-linked antibodies, forming a sandwich complex. A chromophore or enzyme substrate may be added, and color may be allowed to develop. In one embodiment, the color reaction may be stopped, and the color may be quantified using, for example, a spectrophotometer, and/or the color may be subjectively assessed by the human eye. Presence of the signal at the target reaction zones indicates the presence of the Bordetella bronchiseptica antigen in the sample of the biological fluid obtained from the animal. Presence of the signal at the control zone indicates that the assay is working properly and that canine samples have been added. The presence of the signal may indicate an active Bordetella bronchiseptica infection in the animal as shown in FIG. 1.

### Example 3: Evaluation of a Bordetella bronchiseptica Infection Outbreak in a Dog Kennel

A training center for racing greyhound dogs is observing that several dogs are showing symptoms of respiratory discomfort. The cause is unknown and a method for rapidly evaluating the possibility of a kennel cough outbreak is needed.

Oral swabs from each of the dogs in the center is obtained and marked with identification information as necessary. The samples are added to a multiplexed assay device that can detect the presence of a Bordetella bronchiseptica infection as shown in FIG. 5. The device contains multiple assay strips attached to a radial cassette unit in a hub-spoke fashion, wherein each assay strip is numerically identified. The samples are added to the assay strips in the cassette unit according to the sample identification numbers. Each assay strip comprises antibodies specific to Bordetella bronchiseptica pertactin and FHA in two distinct target reaction zones as shown in FIG. 3 and are attached to the substrate of the assay strip. In the next step HRP-linked antibodies such as anti-Per-HRP and anti-FHA-HRP are added to specifically bind to one or more of the Pertactin and FHA antigens that may be present in one or more of the samples obtained from the animal in the training center. The attached antibodies at the target reaction zones (anti-Per and anti-FHA) bind to the antigens (Per and FHA respectively) in one or more of the samples. The antigens are also bound to the HRP-linked antibodies, forming a sandwich complex.

A chromophore or enzyme substrate is added, and color allowed to develop which takes about 5-10 minutes. The color reaction is stopped, and the color is quantified using a smart phone, and/or the color is subjectively assessed by the human eye. The data is processed and based on the signal intensity in each assay strip (indicates the presence of Bordetella bronchiseptica specific antigens), Bordetella bronchiseptica infection is diagnosed rapidly in the sample of the biological fluid obtained from the animal corresponding to the numerical identifier. Rapid evaluation of the status of Bordetella bronchiseptica infection among the dogs in the training center would help to arrest the spread of a possible kennel cough outbreak.

### Example 4: Development of an Application for Diagnosis and Analysis Via Inspection of Lateral Flow Test Results

In this example, a ControlPoint Application is developed to provide a quick, reliable diagnosis through inspection of the proprietary, lateral flow test. As more tests are performed, the database supporting the application and storage of test results is developed to enable large scale data analysis.

### User Interface

Once a user visits the ControlPoint Application, a unique profile is created tying them to a specific clinic and allowing all future tests to be recalled as desired. Standard contact information and authorizations to enable location services is requested to assist with long-term data analysis and convenience of use.

During a given test, an array of color-based indicators unique to each potential diagnosis is photographed and uploaded to ControlPoint's, cloud-based database. Once received, an artificially intelligent algorithm inspects the image to locate device traceability information along with the results for each unique pathogen. If enabled, location and environmental data is stored along with all standard test data such as patient, owner, technician, veterinarian, and clinic information. Upon completion of analysis, the user is prompted to upload an additional image—in the case of poor image quality—or provided with results. All data is capable of being exported, sent via email, or recalled through the application at any time for additional review.

As more tests are performed, the regional data is analyzed to track trends tied to location or environmental conditions and correlated to prevalence of specific pathogens. This function is isolated to the administrators of ControlPoint, ensuring the security of users.

### Database Structure

Next, a database is built using a MySQL structure consisting of 3 unique tables. The first table is designed to contain clinic information. As more users at different locations begin using the ControlPoint Application, unique clinic profiles are created. By doing so, tests taken by users at a clinic are tracked, allowing clinic-based analysis. Table 2 describes each unique input intended to be collected upon initialization.

TABLE 2
Clinic Information

| Data Field | Data Type | Modifier | Notes |
|---|---|---|---|
| Id | INT | PRIMARY KEY | Automatically |
| | | | assigned from a |
| | | | unique set of id's |
| | | | To have a unique |
| | | | GUEST clinic to |
| | | | account for offline, |
| | | | incorrect entries |
| Name | VARCHAR(255) | NOT NULL | Input by user |
| Address1 | VARCHAR(255) | NOT NULL | Input by user |
| Address2 | VARCHAR(255) | | Input by user |
| City | VARCHAR(255) | NOT NULL | Input by user |
| State | VARCHAR(255) | NOT NULL | Input by user |
| Zip Code | INT | NOT NULL | Input by user |
| Date | VARCHAR(15) | NOT NULL | Automatically input |
| Registered | | | based on server |
| | | | current date |
| Primary | INT | NOT NULL | Automatically |
| Physicians | | | updated when new |
| Registered | | | physician registers |
| | | | through the app |
| Technicians | INT | NOT NULL | Automatically |
| Registered | | | updated when new |
| | | | technician registers |
| | | | through the app |
| Total Tests | INT | NOT NULL | Automatically |
| | | | updated when new |
| | | | test is uploaded |

Next, User specific data is tracked in a second SQL table. Tests taken by an individual are also be tied to their unique identity. See Table 3 for user data to be stored.

TABLE 3
User Information

| Data Field | Data Type | Modifier | Notes |
|---|---|---|---|
| Id | INT | PRIMARY | Automatically |
| | | KEY | assigned from a |
| | | | unique set of id's |
| | | | To have a unique |
| | | | GUEST user to |
| | | | account for offline, |
| | | | incorrect entries |
| Name | VARCHAR(255) | NOT NULL | Input by user |
| Password | VARCHAR(255) | NOT NULL | Input by user |
| Position | INT | NOT NULL | Options provided to |
| | | | user as primary, tech, |
| | | | or other, assigned by |
| | | | enumeration (0 = |
| | | | n/a, 1 = physician, |
| | | | 2 = tech) |
| Credentials | VARCHAR(255) | | Input by user |
| Associated | INT | | Selected by user |
| Clinic | | | upon registration, |
| | | | FOREIGN KEY |
| | | | assigned according |
| | | | to clinic id |
| Date | VARCHAR(15) | NOT NULL | Automatically input |
| Registered | | | based on server |
| | | | current date |
| Contact phone | BIGINT | | Input by user |
| Contact email | VARCHAR(255) | NOT NULL | Input by user |
| Total Tests | INT | NOT NULL | Automatically |
| | | | updated when new |
| | | | test is uploaded |

Finally, an SQL table is designed to track the unique conditions and results of each test performed. Table 4 describes data gathered for each test performed.

TABLE 4
Test Data

| Data Field | Data Type | Modifier | Notes |
|---|---|---|---|
| Id | INT | PRIMARY | Automatically |
| | | KEY | assigned from a |
| | | | unique set of id's |
| Tester | INT | | Automatically input |
| | | | based on current user, |
| | | | FOREIGN KEY |
| | | | assigned according to |
| | | | user id |
| Test Date | VARCHAR(15) | NOT NULL | Automatically input |
| | | | based on device |
| | | | current date |
| Upload Date | VARCHAR(15) | NOT NULL | Automatically input |
| | | | based on server |
| | | | current date |
| Test Clinic | INT | | Automatically input |
| | | | based on current |
| | | | clinic, FOREIGN |
| | | | KEY assigned |
| | | | according to clinic id |
| Pet Owner | VARCHAR(255) | NOT NULL | Input by user |
| Patient Name | VARCHAR(255) | NOT NULL | Input by user |
| Patient Breed | VARCHAR(255) | NOT NULL | Input by user |
| Patient age | TINYINT | NOT NULL | Input by user |
| | UNSIGNED | | |
| Symptoms | VARCHAR(10000) | NOT NULL | Automatically input |
| | | | based on file location |
| | | | where text is stored |
| Sample Type | TINYINT | NOT NULL | Options provided to |
| | UNSIGNED | | user as oral, |
| | | | conjunctival sample, |
| | | | nasal, etc, assigned |
| | | | by enumeration |
| Image | VARCHAR(10000) | NOT NULL | Automatically input |
| | | | based on file location |
| | | | where image is stored |
| Device Used | TINYINT | NOT NULL | Options provided to |
| | UNSIGNED | | user as |
| | | | DeviceName1, |
| | | | DeviceName2, etc, |
| | | | assigned by |
| | | | enumeration |
| Device Lot | VARCHAR(255) | NOT NULL | Automatically input |
| | | | based on image |
| | | | recognition |
| Test Results | INT | NOT NULL | Automatically input |
| | | | based on image |
| | | | recognition, assigned |
| | | | by binary |
| | | | enumeration |
| Test | VARCHAR(255) | | Automatically input |
| Location | | | based on device |
| | | | coordinates |
| | | | If GUEST, input by |
| | | | user according to city |
| Test Time | VARCHAR(15) | NOT NULL | Automatically input |
| | | | based on device |
| | | | current time |
| Test | VARCHAR(15) | | Input by user |
| Location | | | |
| Temperature | | | |
| Regional | VARCAHR(15) | | Automatically input |
| Temperature | | | based on local |
| | | | weather reading |

### Back-End Structure

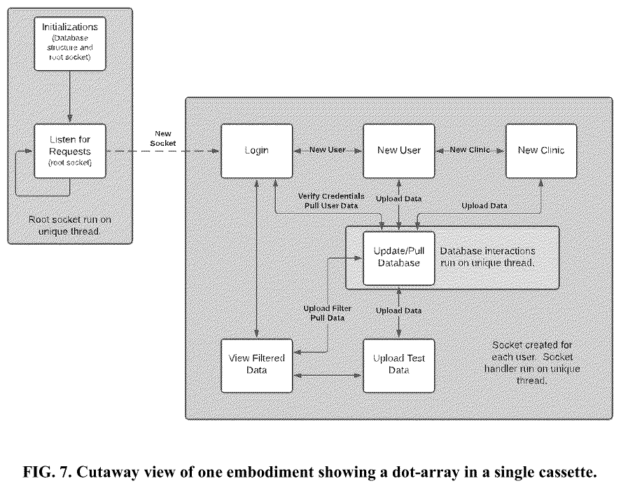

In order to facilitate multiple simultaneous users, the multithreaded, Python-based back-end is broken up into three high level functions. FIG. 7 describes the interactions between each unique thread. Incoming requests and outgoing responses are handled by a unique thread. Each request enters a queue that is serviced in a first-in, first-out method. Once routed to and while processing through the appropriate function, the thread is freed up to service additional requests. As a previous response to a request is formed, a flag is raised allowing the response to be returned to the source.

Unique functions are called depending on the request type. Execution of each function occurs on an additional thread. Acting as a bridge between requests and database interactions, this thread uploads or pulls data in the necessary order to ensure new users and clinics are created in the appropriate manner, existing users can log in, new tests can be processed, and previous tests can be visualized. This allows each request to be serviced and provides sufficient processing time without slowing down application response time as more users are active. Depending on the required processing needed to analyze a given test result, an additional thread may be created to further avoid delays. Modifications and reading from the database occur on a final thread. This thread manages all changes to the database to avoid data corruption due to multiple functions accessing simultaneously.

### Front-End Structure

Users gain access to ControlPoint's application features through a visually friendly user interface. A web-based application implements HTML, JavaScript, and CSS to collect information, send requests, receive responses, and display data provided from the back-end. As shown in FIG. 7, a user must first either login or create a new profile. If they are unable to locate their associated clinic from the list provided, a new clinic profile is created.

Upon approval of login credentials, a user has the option of viewing previous tests taken by themselves and their associated clinic or performing an additional test. When reviewing previous test data, filtering options are made available by date, breed, or test results. This enables one to quickly navigate to the test, or series of tests, of interest and export/send data as desired. Alternatively, a user is given the option to perform a new test by following the instructions demonstrated on the application, uploading a photo of the device in which fluid samples were deposited, and receiving feedback from the application's analysis algorithm. In enabled, location and temperature settings are automatically gathered and displayed to the user to assist in their analysis.

While the web-based application is compatible with any device with the ability to connect to the internet, additional applications are to be created for common operating systems enabling offline functionality. Additional languages such as Swift and Android (Java) are added to front-end capabilities.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for processing or analyzing a sample from a subject in need thereof, the method comprising:
a) processing said sample using a lateral flow device configured to specifically bind to one or more target analytes associated with a respiratory condition associated with one or more pathogens that can cause Canine Infectious Respiratory Disease Complex (CIRDC) in one or more discrete sections of said lateral flow device,
wherein said lateral flow device is configured to produce a detectable signal in said one or more discrete sections upon binding to said one or more target analytes; and
b) analyzing said detectable signal to determine if said subject has said respiratory condition or has an immunity to said respiratory condition.

2. The method of claim 1, further comprising obtaining said sample from said subject via swab.

3. The method of claim 1, wherein said sample is selected from the group consisting of saliva, ocular discharge, nasal discharge, oropharyngeal fluid, oral rinse expectorant, oral fluid, gingival cervicular fluid, urine, sweat, tears, blood, serum, stool, gastric fluid, synovial fluid, and phlegm.

4. The method of claim 1, wherein said target analytes comprise one or more proteins.

5. The method of claim 4, wherein said one or more proteins is an antigen produced by a CIRDC pathogen, a virulence factor produced by said CIRDC pathogen, a fragment of a virulence factor produced by said CIRDC pathogen, said CIRDC pathogen or an immunogenic fragment thereof.

6. The method of claim 5, wherein said CIRDC pathogen is selected from the group consisting of Bordetella bronchiseptica; Canine parainfluenza virus; Canine coronavirus; Canine adenovirus type-2; Canine herpesvirus; Canine distemper virus; Canine Influenzavirus; Canine Pneumovirus; Mycoplasma cynos; Streptococcus equi; Canine bocavirus; and Canine hepacivirus.

7. The method of claim 4, wherein said antigen is a virulence factor of one or more of: Bordetella bronchiseptica, canine parainfluenza virus, canine respiratory coronavirus, canine adenovirus type-2, canine herpesvirus, canine distemper virus, canine influenza virus, canine pneumo virus, Mycoplasma cynos, Streptococcus equi, and canine bocavirus.

8. (canceled)

9. The method of claim 4, wherein said target analytes comprise a target antibody.

10. The method of claim 9, wherein said target antibody comprise an antibody with affinity to one or more immunogenic components of a CIRDC pathogen.

11. The method of claim 10, wherein said target antibody is selected from the group consisting of: Bordetella bronchiseptica, canine parainfluenza virus, canine respiratory coronavirus, canine adenovirus type-2, canine herpesvirus, canine distemper virus, canine influenza virus, canine pneumo virus, Mycoplasma cynos, Streptococcus equi, and canine bocavirusproteins.

12. (canceled)

13. The method of claim 12, wherein a vaccination status of said subject for said respiratory condition is unknown.

14. The method of claim 13, wherein said subject is a non-human animal, wherein said subject belongs to a genus selected from the group consisting of Canis familiaris, Sus scrofa domesticus, Felis catus, Oryctolagus cuniculus and Equus caballus.

15. (canceled)

16. (canceled)

17. The method of claim 1, wherein said respiratory condition is an infectious tracheobronchitis.

18. The method of claim 17, wherein said infectious tracheobronchitis is a Bordetella bronchiseptica, Canine parainfluenza virus, Canine coronavirus, Canine adenovirus type-2, Canine herpesvirus, Canine distemper virus, Canine Influenza virus, Canine Pneumovirus, Mycoplasma cynos, Streptococcus equi, Canine bocavirus or Canine hepacivirus infection.

19. The method of claim 17, wherein said infectious tracheobronchitis is a viral infection or a bacterial infection.

20. (canceled)

21. The method of claim 1, further comprising evaluating an onset of protective immunity to said respiratory condition in said subject that has received a vaccine to said respiratory condition.

22. The method of claim 1, wherein analyzing comprises comparing said detectable signal to a control to determine a presence of said target analytes in said sample.

23. The method of claim 22, wherein said comparing comprises analyzing a digital image of said detectable signal produced by said lateral flow device, wherein said digital image comprises a code to identify said control.

24. The method of claim 23, wherein said analyzing is performed by a trained algorithm, wherein said digital image is sent to said trained algorithm via a computer.

25. The method of claim 1, comprising obtaining a second sample from said subject at a second time point and analyzing said second sample using said lateral flow device to evaluate a duration of immunity to said respiratory condition.

26.-71. (canceled)
